(19) United States
(12) Patent Application Publication
(10) Pub. No.: US 2025/0369981 A1
(43) Pub. Date: Dec. 4, 2025

(54) Phenylglyoxal-Based Alkyne (PGA) Chemical Tag for Protein Citrullination Analysis

(71) Applicant: Wisconsin Alumni Research Foundation, Madison, WI (US)

(72) Inventors: Lingjun Li, Madison, WI (US); Miyang Li, Vista, CA (US); Min Ma, San Diego, CA (US); Hung-Yu Chiang, Madison, WI (US)

(21) Appl. No.: 19/227,084

(22) Filed: Jun. 3, 2025

Related U.S. Application Data
(60) Provisional application No. 63/655,869, filed on Jun. 4, 2024.

(51) Int. Cl. G01N 33/68; G01N 33/532

(52) U.S. Cl. CPC G01N 33/6848; G01N 33/532; G01N 2440/18; G01N 2458/15

(57) ABSTRACT

The present invention provides phenylglyoxal-based alkyne (PGA) chemical tags exhibiting high specificity towards protein citrullination sites and other biomolecules containing similarly reactive functional groups. The PGA tags are able to bind to or derivatize biomolecules, such as polypeptides having one or more post-translational modifications (PTMs), such as citrullination. In particular, the PGA tags of the present invention have superior reactivity and selectivity towards ureido groups, and allow for the analysis of biomolecules containing ureido groups facilitated by click chemistry and mass spectrometry (MS) techniques and methods for qualitative and quantitative analysis of biological and clinical samples.

### CROSS-REFERENCE TO RELATED APPLICATIONS

This application claims priority from U.S. Provisional Patent Application No. 63/655,869, filed Jun. 4, 2024, which is incorporated by reference herein to the extent that there is no inconsistency with the present disclosure.

### STATEMENT REGARDING FEDERALLY SPONSORED RESEARCH OR DEVELOPMENT

This invention was made with government support under R01AG052324 awarded by the National Institutes of Health. The government has certain rights in the invention.

### REFERENCE TO AN ELECTRONIC SEQUENCE LISTING

The contents of the electronic sequence listing (41-24_seq_list.xml; Size: 5,414 bytes; and Date of Creation: Jun. 3, 2025) is herein incorporated by reference in its entirety.

### BACKGROUND OF THE INVENTION

Post-translational modifications (PTMs) are biochemical modifications of proteins following ribosomal translation to form the mature protein, and can be induced by the cleavage of peptide bonds, the formation of disulfide bonds, the modification of existing functional groups, and the addition of new functional groups. Examples of PTMs include, but are not limited to, acetylation, amidation, citrullination, glycosylation, lipidation, methylation, nitrosylation, phosphorylation, proteolysis, and ubiquitination.

PTMs play an important role in the regulation of protein folding, translocation, protein-protein interactions, as well as protein physiological functions. It has been revealed that abnormal alteration of many protein PTMs are associated with the onset and progression of many devastating diseases. Hence, comprehensive and systematic profiling of the dynamic changes of disease-related protein PTMs is not only critical to unravel the pathogenesis of diseases, but beneficial to the diagnosis and treatment of diseases in clinical applications.

Protein citrullination is a PTM that entails the conversion of peptidyl-arginine to peptidyl-citrulline, often catalyzed by a family of calcium-dependent enzymes known as protein arginine deiminases (PADs). This modification exerts a profound influence on protein structural conformation and functionality and has been implicated in various pathological conditions, including rheumatoid arthritis, multiple sclerosis, and Alzheimer's disease. Recent advancements in chemical probe design targeting citrullination have significantly augmented comprehension of these processes. However, the current understanding about protein citrullination is still rather limited and impeded, primarily due to the lack of effective analytical methods for the large-scale analysis of citrullinated proteins in complex biological samples.

Current citrullination studies mainly rely on conventional antibody-based techniques such as enzyme-linked immunosorbent assay (ELISA), immunohistochemistry (IHC), and Western blotting (WB). However, current antibodies either target a specific citrullinated protein or are applied to general protein citrullination analysis. Precise and high-throughput identification of low-abundance citrullinated proteins in complex biological samples is still not readily achievable. In addition, information in terms of protein citrullination localization cannot be revealed by these antibody-based detection methods.

Additionally, while mass spectrometry (MS)-based bottom-up proteomics has been proven to be a useful tool for large-scale-analysis of many important PTMs, such as protein phosphorylation, methylation and glycosylation, its application to protein citrullination analysis suffers from several challenges. For example, conventional methods attempting specific capture of citrullinated peptides for MS analysis are generally not effective, and the mass shift of 0.984 Da induced by citrullination is the same as that of another modification called deamidation. Furthermore, 13C isotopic peaks in tandem MS spectra have a mass shift of 1.0033 Da, which is close to that of citrullination. Each of these may interfere with the accurate identification of peptide fragment ions, leading to misidentification of citrullination sites.

### BRIEF SUMMARY OF THE INVENTION

To address these challenges, the present invention discloses phenylglyoxal-based tags (also referred to herein as “probes”) for the chemical derivatization of biomolecules containing a ureido group, such as those found in citrullination post-translational modifications (PTMs), or a similarly reactive functional group. Also disclosed are methods that integrate chemical derivatization using phenylglyoxal-based tags with mass spectrometry (MS)-based technology for the large-scale and high-confident identification of citrullinated and similarly modified biomolecules. Additionally, the methods are able to be combined with multiple MS-based quantitative strategies, including isotopic and isobaric tag labeling techniques, enabling the concurrent qualitative and quantitative analysis of citrullinated and similarly modified biomolecules from various biological samples.

In an embodiment, the phenylglyoxal-based tags of the present invention have superior reactivity and selectivity towards ureido groups under acidic and highly acidic conditions, including ureido groups present in citrullination, carbamylation, and homocitrullination. Analysis of biomolecules containing ureido groups and protein citrullination, carbamylation, and homocitrullination using these tags are further facilitated by click chemistry and mass spectrometry (MS) techniques.

In an embodiment, the present invention provides a phenylglyoxal-based alkyne (PGA) chemical tag having high reaction specificity towards biomolecules containing PTM sites, ureido groups, and similar functional groups. In an embodiment, the biomolecule is a polypeptide, especially a polypeptide which has undergone post-translational modification, including, but are not limited to, acetylation, acylation, alkylation, amidation, carbamylation, citrullination, glycosylation, hydroxylation, iodination, lipidation, methionine oxidation, methylation, nitrosylation, phosphorylation, prenylation, sulfonation, neddylation, SUMOylation, and ubiquitination. Preferably, the polypeptides are polypeptides that have undergone citrullination, carbamylation, homocitrullination, and combinations thereof.

In an embodiment, the present invention provides a method for the analysis of a target biomolecule comprising the steps of: a) providing a sample containing the target biomolecule, where the target biomolecule comprises a polypeptide having a region modified by a post-translational modification (PTM); and b) mixing a phenylglyoxal-based alkyne (PGA) tag with the target biomolecule to generate a labeled biomolecule. The PGA tag is able to generate a derivatized polypeptide from the polypeptide having the region modified by the PTM, and imparts a mass shift to the target biomolecule.

However, the present invention is not limited to polypeptides and may be applied to other biomolecules containing a ureido group or a similar functional group. In an embodiment, the method comprises a) providing a sample containing the target biomolecule, wherein the target biomolecule comprises a ureido group; and b) mixing a phenylglyoxal-based alkyne (PGA) tag with the target biomolecule to generate a labeled biomolecule. Again, adding the PGA tag with the target biomolecule imparts a mass shift to the target biomolecule.

The above methods may additionally comprise ionizing the labeled biomolecule to form a precursor ion; detecting and analyzing the precursor ion using a mass spectrometer; and identifying biomolecules with mass spectrometry data.

Additionally, the labeled biomolecule comprises an alkyne moiety and the methods described herein optionally further comprise adding an additional functional tag to the labeled biomolecule, wherein the additional functional tag comprises an azide group. The alkyne moiety and azide group are able to form a reaction that results in the functional tag being added to the labeled biomolecule. Such functional tags include, but are not limited to biotin tags comprising an azide group, such as a DADPS-Biotin-Azide functional tag or a DiLeu-Biotin-Azide (cDBA) functional tag. The functional tag may further be attached to a solid support, such as a resin bead or magnetic bead.

In an embodiment, the additional functional tag is an isotopically enriched functional tag comprising one or more heavy isotopes present in an amount in excess of the natural isotopic abundance. The isotopically enriched functional tag can be any of the tags or functional tags described above, wherein any number of carbons in the tagging reagent are ¹²C or ¹³C, ¹⁴N or ¹⁵N, ¹H or ²H; and ¹⁶O or ¹⁸O. Optionally, the biomolecule is a citrullinated polypeptide and the isotopically enriched functional tag is an isotopically enriched dimethyl tag. Optionally, the isotopically enriched functional tag is a tandem mass tag (TMT), dimethyl leucine tag (DiLeu), or iTRAQ tag (Boersema et al., Nat. Protoc., 2009, 4 (4): 484-494; Frost et al., Anal. Chem., 2017, 89 (20): 10798-10805; Frost et al., Anal. Chem., 2015, 87 (3): 1646-1654; and Frost et al., Anal. Chem. 2020, 92:8228-8234).

In an embodiment, two or more samples containing the target biomolecule are provided, where the PGA tag is added with the target biomolecule to generate a labeled biomolecule in each of the samples. A different isotopically enriched functional tag is added to the labeled biomolecule in each sample, thereby generating two or more samples comprising different isotopically labeled target biomolecules. The different isotopically labeled target biomolecules from the two or more samples are combined and the mixture is ionized to form precursor ions. Precursor ions from each of the two or more samples are detected and analyzed using a mass spectrometer. In an embodiment, 2, 3, 4, 5, 6, 7, 8, 9, 10 or more different samples are reacted with different isotopically enriched functional tags.

In an embodiment, the PGA tag and/or the additional functional tag impart a mass shift to the target biomolecules, which allows the target biomolecules to be more easily identified and distinguished from other molecules, such as a protein having undergone a different PTM. However, the tags may not only change the solubility of tag-labeled biomolecules, but also affect the fragmentation patterns upon tandem mass spectrometry analysis. Thus, it is important to consider the influence on MS-based fragmentation of tag-labeled biomolecules. If the tagging reagent has a mass over 500 Da, higher collisional energy is often required for the efficient fragmentation of these tag-labeled biomolecules. In addition, there may be more than one MS-cleavable sites present. When high collisional energy is used, the PGA tag and/or the additional functional tags themselves will be cleaved, which could suppress the peptide backbone fragmentation and result in a tandem MS spectrum with poor quality. Hence, a small PGA tag and/or additional functional tag with no more than one MS-cleavable site is preferable for MS-based analysis. In an embodiment, reacting the PGA tag and/or the additional functional tag with the target biomolecules imparts a mass shift from approximately 1 Da to 500 Da to the target biomolecules, preferably a mass shift from 10 Da to 400 Da, a mass shift from 20 Da to 350 Da, a mass shift from 20 Da to 250 Da, or a mass shift from 100 Da to 200 Da to the target biomolecules.

In an embodiment, a PGA tags suitable for use in the present invention comprises one or more of:

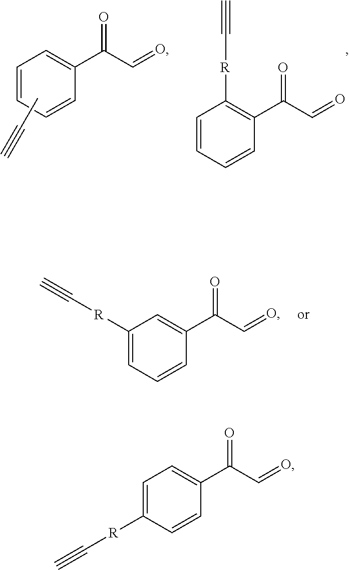

where R is selected from the group consisting of substituted and unsubstituted C₁ to C₂₀ alkylene groups and C₁ to C₂₀ amide groups. Optionally, R is an amide having between 1-10 carbon atoms. Optionally, R is an amide having between 1-6 carbon atoms. Optionally, R is an amide having between 1-4 CH₂ groups.

In an embodiment, the PGA tag comprises one or more of:

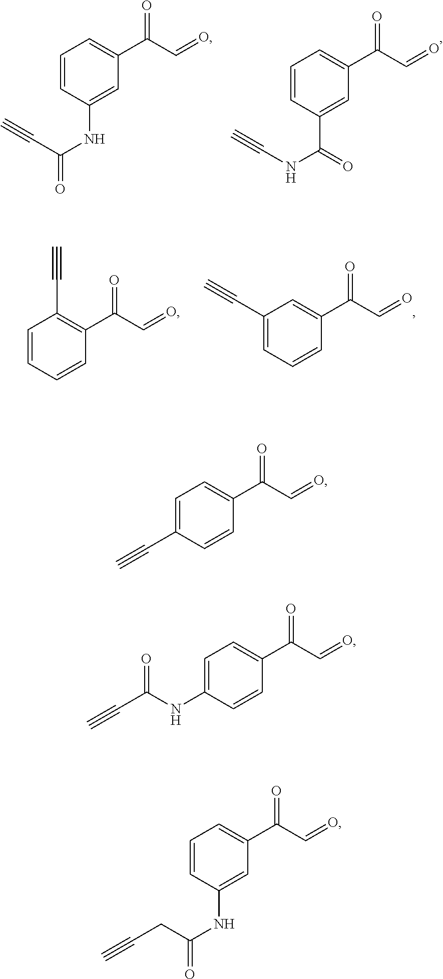

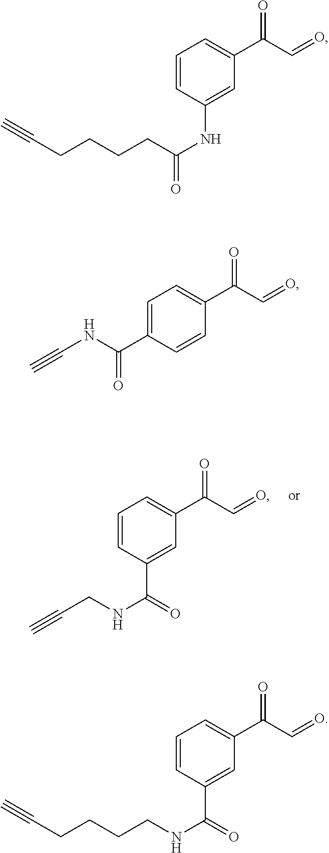 [SMILES: C#CCCCCC(=O)Nc1cccc(C(=O)C=O)c1 ; C#CNC(=O)c1ccc(C(=O)C=O)cc1 ; C#CCNC(=O)c1cccc(C(=O)C=O)c1 ; C#CCCCCNC(=O)c1cccc(C(=O)C=O)c1]

Preferably, the PGA tags exhibit high reaction specificity towards protein citrullination sites, carbamylation sites, and homocitrullination sites, where the alkyne group of the tag is then able to form a reaction with various azide-containing functional tags through click chemistry, thereby facilitating downstream analytical procedures.

In an embodiment, a biomolecule containing a ureido group is reacted with a PGA tag, resulting in the biomolecule being labeled with a portion of the PGA tag having the alkyne moiety. Optionally, the alkyne moiety is then reacted with an additional tagging agent, allowing for diverse analytical approaches of the tagged biomolecule, such as enrichment, immunostaining, fluorescence detection/imaging, and mass spectrometry analysis. Such additional tagging agents include, but are not limited to biotin, biotin-derived tags, and fluorescent labels.

Preferably, the biomolecules are citrullinated proteins or peptides, where the proteins or peptides are subjected to a reaction with a PGA tag under acidic conditions, resulting in the labeling of citrullinated sites with the PGA tag, each tag bearing the alkyne moiety. Optionally, azide-containing functional tags are then conjugated to the labelled citrullination sites, such as through a copper-catalyzed azide-alkyne cycloaddition (CuAAC) reaction. The versatility of the azide-containing functional tags, including the potential incorporation of biotin, fluorescent labels, or signature fragments for mass spectrometry, allows for diverse analytical approaches such as enrichment, immunostaining, fluorescence detection/imaging, and mass spectrometry analysis.

These tags and methods have widespread applications in both fundamental studies of protein citrullination structure and function as well as clinical applications for diagnostics and development of therapeutic targets as the PGA tags enable significant advancements in the understanding of PAD enzyme mediated citrullination in various diseases, ranging from cancer, immunological disorders, and neurodegenerative diseases.

### BRIEF DESCRIPTION OF THE DRAWINGS

FIG. 1 illustrates a post-translational modification (PTM) of arginine to citrulline as catalyzed by protein arginine deiminases (PADs). This PTM results in +0.984 Da on the initial arginine residue and loss of positive charge under physiological conditions.

FIG. 2 illustrates chemical derivatization of citrulline using 2,3-butanedione followed by further addition of a biotin tag (top) or antipyrine to form a COLDER complex (bottom).

FIG. 3 illustrates the chemical derivatization of citrulline using phenylglyoxal and 2,3-butanedione (described in J. Am. Chem. Soc. 2012, 134, 41, 17015-17018; and Curr Opin Chem Biol. 2016 30:1-6).

FIG. 4 illustrates prior phenylglyoxal-based derivatives that do not include the alkyne moiety required by the present invention (described in Curr Opin Chem Biol. 2016 30:1-6).

FIG. 5 shows an embodiment of the present invention, where a peptide containing a citrullinated site is reacted with a PGA tag under acidic conditions (pH<1). The PGA tag is able to react with the ureido group of the citrullinated site with high specificity.

FIG. 6. shows a synthetic route of a PGA tag in an embodiment of the present invention.

FIG. 7 shows various different PGA tags in an embodiment of the present invention.

FIG. 8 shows the 1H-NMR and HPLC-UV-MS spectra of a PGA tag in an embodiment of the present invention. 1H NMR (400 MHZ, DMSO-d₆) δ=11.23-10.90 (m, 1H), 9.50 (s, 0.6 H), 8.42-8.26 (m, 1H), 7.94-7.73 (m, 2H), 7.62-7.40 (m, 1.3 H), 6.76 (d, J=7.3 Hz, 0.8 H), 5.65 (t, J=7.3 Hz, 0.4H), 4.55-4.40 (m, 1H).

FIG. 9 shows the HPLC-UV-MS spectra of the PGA tag of FIG. 8.

FIG. 10 shows MALDI-MS spectra of three different peptide standards reacted with PGA: panel A-SGRGKGGKGLGKG (SEQ ID NO:1), panel B-SAVRARSSVPGVR (SEQ ID NO: 2), and panel C-ARHGGLPRARDG (SEQ ID NO:3). FIG. 10 (panels A and B) shows the spectra of the peptide standards (not citrullinated), the peptide standard (not citrullinated) mixed with the PGA tag, the citrullinated peptide standard where the arginine (R) residues are converted to citrulline (Cit), and the citrullinated peptide standard reacted with the PGA tag. Each example was exposed to the acidic conditions (pH<1). FIG. 10 (panel C) shows a citrullinated peptide standard where the arginine (R) residues are converted to citrulline (Cit), and the citrullinated peptide standard mixed with the PGA tag after 3 hours, 6 hours, and 16 hours.

FIG. 11 (panels A-C) shows ESI-MS/MS fragment spectra of the citrullinated peptides labeled with the PGA tag of FIG. 8: panel A-SGRGKGGKGLGKG (SEQ ID NO:1), panel B-SAVRARSSVPGVR (SEQ ID NO:2), and panel C-ARHGGLPRARDG (SEQ ID NO: 3).

FIG. 12 shows the MS/MS spectrum from FIG. 10 (panel C-ARHGGLPRARDG (SEQ ID NO:3) where the signature immonium ion of the citrullinated peptide labeled with the PGA tag is seen at m/z 313.1292.

FIG. 13 shows an ESI-MS/MS spectrum of a citrullinated peptide of Histone H4, VFLENVIRDAVTYTEHAK (SEQ ID NO:4), where the arginine (R) residue is converted to citrulline, labeled by PGA and further labeled by a DADPS-Biotin-Azide functional tag.

FIG. 14 shows the MS/MS spectrum from FIG. 13 where the signature immonium ion of the citrullinated peptide VFLENVIRDAVTYTEHAK (SEQ ID NO:4) labeled with the PGA and azide tag is seen at m/z 456.

FIG. 15 shows ESI-MS/MS spectrum of the same citrullinated peptide of Histone H4 VFLENVIRDAVTYTEHAK (SEQ ID NO:4) labeled by PGA and further labeled by a cleavable DiLeu-Biotin-Azide (cDBA) tag. The signature immonium ion of the PGA-labeled peptide is not preserved but an abundant amount of the cleaved DiLeu reporter ion is seen at m/z 114.

FIG. 16 provides graphs showing enrichment enhancement achieved by DADPS-Biton-Azide and cDBA tags of PGA-labeled peptides of Histone H4 spiked in a human K562 protein digest. 10 μg of PGA-labeled Histone H4 peptides were mixed with 100 μg of Promega human digestion, followed by additional tagging with DADPS-Biotin-Azide and cDBA tags and streptavidin enrichment. >45× and >69× enrichment enhancement was achieved by the DADPS-Biton-Azide and cDBA tags, respectively.

FIG. 17 shows structures of DADPS-biotin-azide and cDBA tags able to react with the PGA-labeled peptides in an embodiment of the present invention.

FIG. 18 illustrates integration of the PGA labeling with an isobaric chemical platform: isobaric tandem orthogonal proteolysis activity-based protein profiling (isoBOP-ABPP) based on the cDBA tag.

FIG. 19 shows structures of alternative phenylgloxal-based azide probes and prior failed alkylene probes.

FIG. 20 shows the structure of homocitrulline PTM, which also contains a ureido group capable of reacting with the PGA tags of the present invention.

FIG. 21 shows selective reactivity of a PGA tag with citrulline under acidic conditions (pH<1) compared to the peptide standard SGRGKGGKGLGKG (SEQ ID NO:1) and a mixture of the peptide and PGA tag at a pH of 7.6.

FIG. 22 illustrates optimized conditions for probe labeling, determining the ideal probe concentration and labeling time. After a 6-hour incubation period, all three citrullination sites of the peptide ARHGGLPRARDG (SEQ ID NO:3) were fully labeled, resulting in more informative MS2 fragment ions (bottom panel).

FIG. 23 shows 10 μg of citrullinated histone protein, including the fragment RISDLIYEETRGVLK (SEQ ID NO:5), being spiked into 1 mg of a complex proteome to evaluate the enrichment efficiency of the present methods. With only one site not detected, all remaining 13 citrullination sites were successfully identified and quantified. Abundant b,y ions were produced to unambiguously pin-point Cit-sites. The PGA tag and isoBOP-ABPP platform provide the capability for multiple PTM analyses.

FIG. 24 shows western blot analysis of in vitro PAD-catalytic lysates from MDA-MB-231 breast cancer cells for citrullinated proteins and Histone H3 citrullination.

FIG. 25 shows streptavidin-based enrichment (immunoprecipitation) of citrullinated proteins conjugated with PGA via DADPS or cleavable DiLeu-Biotin-Azide (cDBA) tags. The left panel shows a western blot of upregulated citrullination in global proteins and Histone H3 after PAD2-catalysis; after the click chemistry for linkage, Histone H3 blot of IP input indicates equal protein between experiment sets. The right pane shows a biotin IP western blot analysis of different biotin derivation tags (DADPS or cDBA) for enrichment of citrullinated proteins examined by streptavidin and Histone H3 blots.

FIG. 26 shows lysate digests labeled using 4-plex cDBA reagents and mixed at predefined ratios of 1:2:5:10 or in reverse. Samples were analyzed by LC-MS/MS at a resolution of 60,000. The quantification results of citrullination sites analyzed are presented as box-and-whisker plots, showing the median (line), interquartile range (25th-75th percentiles, box), and data spread (5th-95th percentiles, whiskers), demonstrating the accuracy and dynamic range of the PGA and cDBA enabled multiplexed quantification.

### DETAILED DESCRIPTION OF THE INVENTION

### Definitions

In general, the terms and phrases used herein have their art-recognized meaning, which can be found by reference to standard texts, journal references and contexts known to those skilled in the art. The following definitions are provided to clarify their specific use in the context of the invention.

As used herein, the term “analyzing” refers to a process for determining a property of an analyte. Analyzing can determine, for example, physical properties of analytes, such as mass, mass to charge ratio, concentration, absolute abundance, relative abundance, or atomic or substituent composition. In the context of proteomic analysis, the term analyzing can refer to determining the composition (e.g., amino acid sequence, PTM site) and/or abundance of a protein or peptide in a sample.

As used herein, the term “analyte” refers to a compound, mixture of compounds or other composition which is the subject of an analysis. Analytes include, but are not limited to, proteins, modified proteins, peptides, modified peptides, small molecules, pharmaceutical compounds, oligonucleotides, sugars, polymers, metabolites, lipids, and mixtures thereof.

As used herein, the term “mass spectrometry” (MS) refers to an analytical technique for the determination of the elemental composition, mass to charge ratio, absolute abundance and/or relative abundance of an analyte. Mass spectrometric techniques are useful for elucidating the composition and/or abundance of analytes, such as proteins, peptides and other chemical compounds. Mass spectrometry includes processes comprising ionizing analytes to generate charged species or species fragments, fragmentation of charged species or species fragments, such as product ions, and measurement of mass-to-charge ratios of charged species or species fragments, optionally including additional processes of isolation on the basis of mass to charge ratio, additional fragmentation processing, charge transfer processes, etc. Conducting a mass spectrometric analysis of an analyte results in the generation of mass spectrometry data for example, comprising the mass-to-charge ratios and corresponding intensity data for the analyte and/or analyte fragments. Mass spectrometry data corresponding to analyte ion and analyte ion fragments is commonly provided as intensities as a function of mass-to-charge (m/z) units representing the mass-to-charge ratios of the analyte ions and/or analyte ion fragments. Mass spectrometry commonly allows intensities corresponding to different analytes to be resolved in terms of different mass-to-charge ratios. In tandem mass spectrometry (MS/MS or MS2), multiple sequences of mass spectrometry analysis are performed. For example, samples containing a mixture of proteins and peptides can be ionized and the resulting precursor ions separated according to their mass-to-charge ratio. Selected precursor ions can then be fragmented and further analyzed according to the mass-to-charge ratio of the fragments.

As used herein, the term “mass-to-charge ratio” refers to the ratio of the mass of a species to the charge state of a species. The term “m/z unit” refers to a measure of the mass-to-charge ratio. The Thomson unit (abbreviated as Th) is an example of an m/z unit and is defined as the absolute value of the ratio of the mass of an ion (in Daltons) to the charge of the ion (with respect to the elemental charge).

As used herein, the term “mass spectrometer” refers to a device which generates ions from a sample, separates the ions according to mass to charge ratio, and detects ions, such as product ions derived from isotopically enriched compound, isotopic tagging reagents, isotopically labeled amino acids and/or isotopically labeled peptide or proteins. Mass spectrometers include single stage and multistage mass spectrometers, which include tandem mass spectrometers that fragment the mass-separated ions and separate the product ions by mass.

As used herein, the term “precursor ion” is used herein to refer to an ion which is produced during ionization stage of mass spectrometry analysis, including the MS1 ionization stage of MS/MS analysis.

“Fragment” refers to a portion of molecule, such as a peptide. Fragments may be singly or multiply charged ions, and may be derived from bond cleavage in a parent molecule, including site specific cleavage of polypeptide bonds in a parent peptide. Fragments may also be generated from multiple cleavage events or steps. Fragments may be a truncated peptide, either carboxy-terminal, amino-terminal or both, of a parent peptide. A fragment may refer to products generated upon the cleavage of a polypeptide bond, a C—C bond, a C—N bond, a C—O bond or combination of these processes. Fragments may refer to products formed by processes where one or more side chains of amino acids are removed, or a modification is removed, or any combination of these processes. Fragments useful in the present invention include fragments formed under metastable conditions or from the introduction of energy to the precursor by a variety of methods including, but not limited to, collision induced dissociation (CID), surface induced dissociation (SID), laser induced dissociation (LID), electron capture dissociation (ECD), electron transfer dissociation (ETD), or any combination of these methods or any equivalents known in the art of tandem mass spectrometry. Fragments useful in the present invention also include, but are not limited to, x-type fragments, y-type fragments, z-type fragments, a-type fragments, b-type fragments, c-type fragments, internal ion (or internal cleavage ions), immonium ions or satellite ions. The types of fragments derived from an analyte, such as an isotopically labeled analyte, isotopically labeled standard and/or isotopically labeled peptide or proteins, often depend on the sequence of the parent, method of fragmentation, charge state of the parent precursor ion, amount of energy introduced to the parent precursor ion and method of delivering energy into the parent precursor ion. Properties of fragments, such as molecular mass, may be characterized by analysis of a fragmentation mass spectrum.

The terms “peptide” and “polypeptide” are used synonymously in the present description, and refer to a class of compounds composed of amino acid residues chemically bonded together by amide bonds (or peptide bonds). Peptides and polypeptides are polymeric compounds comprising at least two amino acid residues or modified amino acid residues. Modifications can be naturally occurring or non-naturally occurring, such as modifications generated by chemical synthesis. Modifications to amino acids in peptides include, but are not limited to, acetylation, acylation, alkylation, amidation, carbamylation, citrullination, glycosylation, hydroxylation, iodination, lipidation, methionine oxidation, methylation, nitrosylation, phosphorylation, prenylation, sulfonation, neddylation, SUMOylation, ubiquitination, and the addition of cofactors. Peptides include proteins and further include compositions generated by degradation of proteins, for example by proteolytic digestion. Peptides and polypeptides can be generated by substantially complete digestion or by partial digestion of proteins. Polypeptides include, for example, polypeptides comprising 2 to 100 amino acid units, optionally for some embodiments 2 to 50 amino acid units and, optionally for some embodiments 2 to 20 amino acid units and, optionally for some embodiments 2 to 10 amino acid units.

“Protein” refers to a class of compounds comprising one or more polypeptide chains and/or modified polypeptide chains. Proteins can be modified by naturally occurring processes such as post-translational modifications or co-translational modifications. Exemplary post-translational modifications or co-translational modifications include, but are not limited to, citrullination, phosphorylation, glycosylation, lipidation, prenylation, sulfonation, hydroxylation, acetylation, methylation, methionine oxidation, the addition of cofactors, proteolysis, and assembly of proteins into macromolecular complexes. Modification of proteins can also include non-naturally occurring derivatives, analogues and functional mimetics generated by chemical synthesis. Exemplary derivatives include chemical modifications such as alkylation, acylation, carbamylation, iodination or any modification that derivatizes the protein.

Quantitative analysis in chemistry is the determination of the absolute or relative abundance of one, several, or all particular substance(s) present in a sample. For biological samples, quantitative analysis performed via mass spectrometry can determine the relative abundances of peptides and proteins. The quantitation process typically involves isotopic labeling of protein and peptide analytes and analysis via mass spectrometry.

Many of the molecules disclosed herein contain one or more ionizable groups. Ionizable groups include groups from which a proton can be removed (e.g., —COOH) or added (e.g., amines) and groups which can be quaternized (e.g., amines). All possible ionic forms of such molecules and salts thereof are intended to be included individually in the disclosure herein. With regard to salts of the compounds herein, one of ordinary skill in the art can select from among a wide variety of available counterions that are appropriate for preparation of salts of this invention for a given application. In specific applications, the selection of a given anion or cation for preparation of a salt can result in increased or decreased solubility of that salt.

The compounds of this invention can contain one or more chiral centers. Accordingly, this invention is intended to include racemic mixtures, diasteromers, enantiomers, tautomers and mixtures enriched in one or more stereoisomer. The scope of the invention as described and claimed encompasses the racemic forms of the compounds as well as the individual enantiomers and non-racemic mixtures thereof.

As used herein, “isotopically enriched” and “isotopically labeled” refer to compounds (e.g., such as isotopically labeled amino acids, isotopically labeled standards, isotopically labeled analyte, isotopic tagging reagents, and/or isotopically labeled peptide or proteins) having one or more isotopic labels, such as one or more heavy stable isotopes, present in an amount greater than the naturally occurring abundance. An “isotopic label” refers to one or more heavy stable isotopes introduced to a compound, such as isotopically labeled amino acids, isotopically labeled standards, isotopically labeled analyte, isotopic tagging reagents, and/or isotopically labeled peptide or proteins, such that the compound generates a signal when analyzed using mass spectrometry that can be distinguished from signals generated from other compounds, for example, a signal that can be distinguished from other isotopologues on the basis of mass-to-charge ratio. “Isotopically-heavy” refers to a compound or fragments/moieties thereof having one or more high mass, or heavy isotopes (e.g., stable heavy isotopes such as ¹³C, ¹⁵N, ²H, ¹⁷O, ¹⁸O, ³³S, ³⁴S, ³⁷Cl, ⁸¹Br, ²⁹Si, and ³⁰Si).

In an embodiment, an isotopically enriched composition comprises a compound of the invention having a specific isotopic composition, wherein the compound is present in an abundance that is at least 10 times greater, for some embodiments at least 100 times greater, for some embodiments at least 1,000 times greater, for some embodiments at least 10,000 times greater, than the abundance of the same compound having the same isotopic composition in a naturally occurring sample. In another embodiment, an isotopically enriched composition has a purity with respect to a compound of the invention having a specific isotopic composition that is substantially enriched, for example, a purity equal to or greater than 90%, in some embodiments equal to or greater than 95%, in some embodiments equal to or greater than 99%, in some embodiments equal to or greater than 99.9%, in some embodiments equal to or greater than 99.99%, and in some embodiments equal to or greater than 99.999%. In another embodiment, an isotopically enriched composition is a sample that has been purified with respect to a compound of the invention having a specific isotopic composition, for example using isotope purification methods known in the art.

The term “alkyl” refers to a monoradical of a branched or unbranched (straight-chain or linear) saturated hydrocarbon and to cycloalkyl groups having one or more rings. Alkyl groups as used herein include those having from 1 to 20 carbon atoms, preferably having from 1 to 6 carbon atoms. Alkyl groups include small alkyl groups having 1 to 4 carbon atoms. Alkyl groups include medium length alkyl groups having from 4-10 carbon atoms. Alkyl groups include long alkyl groups having more than 10 carbon atoms, particularly those having 10-20 carbon atoms. Cycoalkyl groups include those having one or more rings. Cyclic alkyl groups include those having a 3-, 4-, 5-, 6-, 7-, 8-, 9-, 10-, 11- or 12-member carbon ring and particularly those having a 4-, 5-, 6-, or 7-member ring. The carbon rings in cyclic alkyl groups can also carry alkyl groups. Cyclic alkyl groups can include bicyclic and tricyclic alkyl groups. Alkyl groups are optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group linked to oxygen and can be represented by the formula R—O. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups.

As used herein, the term “alkylene” refers to a divalent radical derived from an alkyl group or as defined herein. Alkylene groups in some embodiments function as attaching and/or spacer groups in the present compositions. Compounds of the present invention include substituted and unsubstituted C₁-C₃₀ alkylene, C₁-C₁₂ alkylene and C₁-C₅ alkylene groups. The term “alkylene” includes cycloalkylene and non-cyclic alkylene groups.

The term “alkyne” refers to a monoradical of a branched or unbranched unsaturated hydrocarbon group having one or more triple bonds. Alkyne groups include those having from 2 to 20 carbon atoms, preferably having from 2 to 12 carbon atoms, having from 2 to 4 carbon atoms, or having just the 2 carbon atoms involved in the triple bond.

Optional substitution of any alkyl groups includes substitution with one or more of the following substituents: halogens, —CN, —COOR, —OR, —COR, —OCOOR, —CON(R)₂, —OCON(R)₂, —N(R)₂, —NO₂, —SR, —SO₂R, —SO₂N(R)₂ or —SOR groups. Optional substitution of alkyl groups includes substitution with one or more alkenyl groups, aryl groups or both, wherein the alkenyl groups or aryl groups are optionally substituted. Corresponding substitutions may be made for alkylene groups where the substituents are modified to be part of a divalent radical, e.g., —CN—, —OR—, —CON(R)₂—, etc.

As used herein, the term “amide” refers to a hydrocarbon group with the formula R—C(═O)—NR′R″, where R′ is a hydrogen or an alkyl group or an aryl group and more specifically where R′ is a methyl, ethyl, propyl, butyl, or phenyl group, all of which may be optionally substituted.

As used herein, the term “azide” refers to a class of chemical compounds containing three nitrogen atoms as a group, represented by the structure —N═N+=N—.

As used herein, the term “ureido group” refers to a class of chemical compounds containing the univalent radical NH₂CONH—.

### Overview

Post-translational modifications (PTMs) are involved in many serious diseases. However, owing to the lack of effective methods for analyzing many post-translationally modified proteins, such as citrullinated proteins, comprehensive study of such modifications is an as-yet-unresolved challenge.

The present invention provides novel phenylglyoxal-based alkyne (PGA) tags and a multi-faceted method combining chemical derivatization of PTM sites with mass spectrometry (MS)-based technologies for the qualitative and quantitative analysis of target biomolecules, particularly post-translationally modified proteins from complex biological samples. The PGA chemical tags exhibit high specificity towards protein citrullination sites and other biomolecules containing similarly reactive functional groups.

Unlike existing phenylglyoxal-based probes that rely on incorporating an azide group in the tag, the present PGA tags incorporate an alkyne moiety. The PGA tags are able to react with high specificity with the ureido group found in citrullinated proteins and other biomolecules. As a result of this enhanced specificity, the PGA tag is able to significantly advance the understanding of PTMs, including PAD enzyme mediated citrullination in various diseases ranging from cancer (breast cancer), immunological disorders (rheumatoid arthritis), and neurodegenerative diseases (e.g., Alzheimer's disease). The alkyne moiety of the PGA tag also allows high compatibility with biotin tag-assisted enrichment strategies.

The PGA tags have been successfully synthesized and have had their capabilities demonstrated with selectivity studies, enrichment studies, and experiments combining the PGA tags with other tagging reagents, including DiLeu-Biotin-Azide (cDBA) tags.

The PGA tags of the present invention are applicable as a research tool for fundamental studies of PTMs, particularly protein citrullination structure and function. Additionally, these tags are applicable in developing clinical diagnostics and in the identification/development of therapeutic targets.

### EXAMPLES

### Example 1—Development of a Phenylglyoxal-Based Alkyne (PGA) Chemical Tag for Protein Citrullination Analysis Enabled by Click Chemistry and Mass Spectrometry

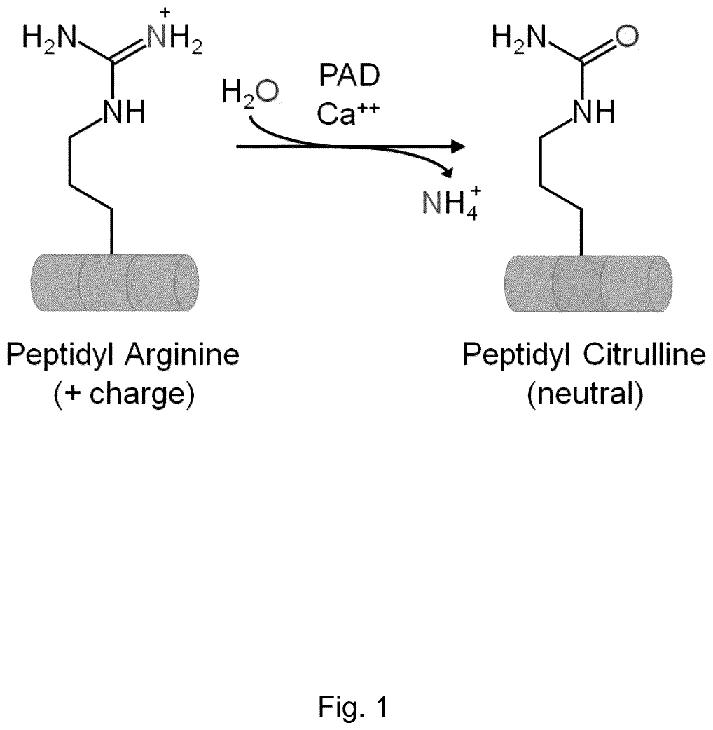

Protein citrullination, a post-translational modification (PTM), entails the conversion of peptidyl-arginine to peptidyl-citrulline, catalyzed by a family of calcium-dependent enzymes known as protein arginine deiminases (PADs) (FIG. 1). This modification exerts a profound influence on protein structural conformation and functionality and has been implicated in various pathological conditions including rheumatoid arthritis, multiple sclerosis, and Alzheimer's disease. Recent advancements in chemical probe design targeting citrullination have significantly augmented comprehension of these processes.

Current citrullination studies mainly rely on conventional analytical techniques, such as enzyme-linked immunosorbent assay (ELISA), immunohistochemistry (IHC), Western blotting (WB), and immunoprecipitation (IP) applications. Such antibody-based techniques provide high specificity towards specific proteins or enable general citrullination level assessment, but cannot provide citrullination-site (Cit-site) information. Additionally, binding efficiency may vary among different vendors and lots. Other analytical techniques, such as COlor DEvelopment Reagent (COLDER) assays and fluorescence-based techniques have similar drawbacks in that they have low sensitivity or cannot provide Cit-site information. Direct analysis using mass spectrometry is challenging due to the small change in mass resulting from the citrullination PTM and interferences with other modifications, such as deamidation. However, if a suitable tag or probe could be developed for citrullination, mass spectrometry analysis could provide Cit-site info and achieve global, large-scale, in-depth analysis.

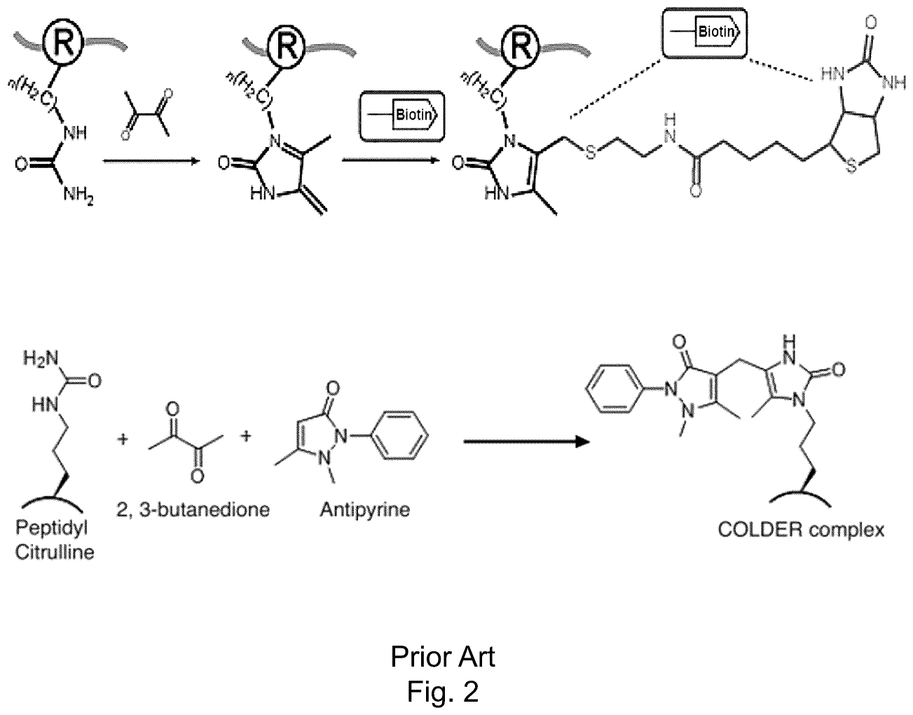

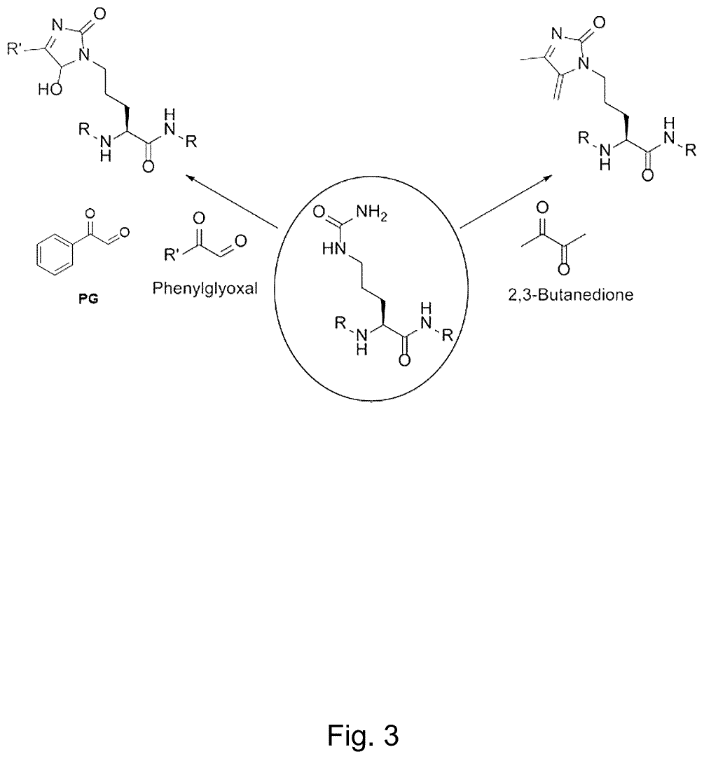

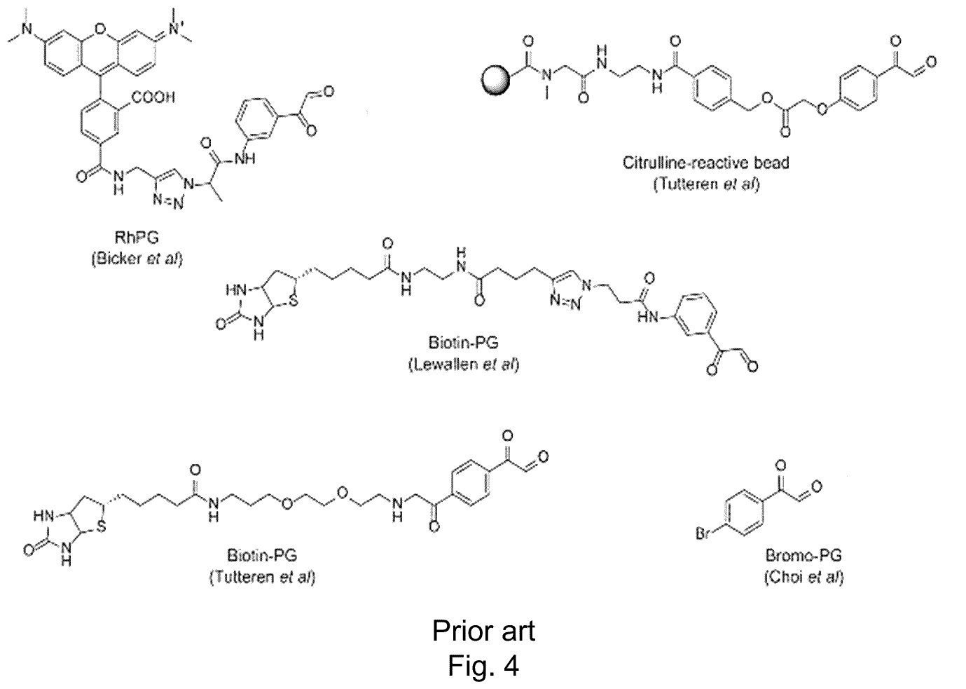

As illustrated in FIG. 2, previous studies have tagged citrulline with 2,3-butanedione followed by further addition of a biotin tag or antipyrine; however, these methods require the use of a three-component reaction, a high nucleophilic thiol biotin, or harsh elution conditions (U.S. application Ser. No. 17/121,242; Anal. Chem. 2022, 94, 51, 17895-17903, Analytical Biochemistry 2000, 286, 257-264; Analytical Biochemistry 2006, 352, 68-76; Current Opinion in Structural Biology 2019, 59:205-215). As a result, phenylglyoxal-based tags have gained substantial attention owing to their reactivity and selectivity towards the ureido group of citrulline under highly acidic conditions (FIGS. 3 and 4). However, prior attempts to utilize phenylglyoxal-based tags have a disadvantage in that it can be difficult or cumbersome to add additional functional tags for analysis or enrichment.

Accordingly, there is a growing interest in leveraging phenylglyoxal derivatives and broadening their applicability. Presented herein is the development of phenylglyoxal-based alkyne (PGA) chemical tags for the analysis of protein citrullination and other biomolecules having an ureido group or a similar functional group able to react with the phenylglyoxal-based tag. These PGA chemical tags exhibit a high specificity towards protein citrullination sites and offer compatibility with various additional functional tags, such as those containing azide functional groups through click chemistry, thereby facilitating downstream mass spectrometry techniques and other analytical procedures.

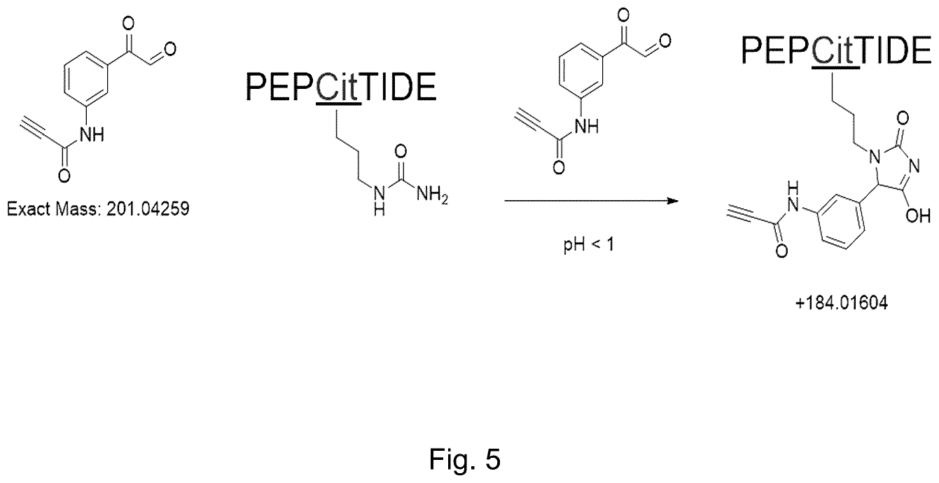

In one method, proteins or peptides containing a citrullinated site are subjected to reaction with PGA under acidic conditions, resulting in the labeling of the citrullinated sites with PGA, each bearing an alkyne moiety (see FIG. 5). Subsequently, azide-containing functional tags are conjugated to these citrullination sites via copper-catalyzed azide-alkyne cycloaddition (CuAAC) reaction (see Fantoni, N Z. et al. Chem Rev. 2021, 121 12:7122-7154; and Parker C G.; Pratt M R. Cell. 2020, 180 605-632). These reactions are high yielding and wide in scope, and create byproducts that can be removed without chromatography. Additionally, the CuAAC reaction is stereospecific, simple to perform, and can be conducted in easily removable or benign solvents.

The versatility of the azide-containing functional tags, including the potential incorporation of biotin, fluorescent labels, or signature fragments for mass spectrometry, allows for diverse analytical approaches such as enrichment, immunostaining, fluorescence detection/imaging, and mass spectrometry analysis.

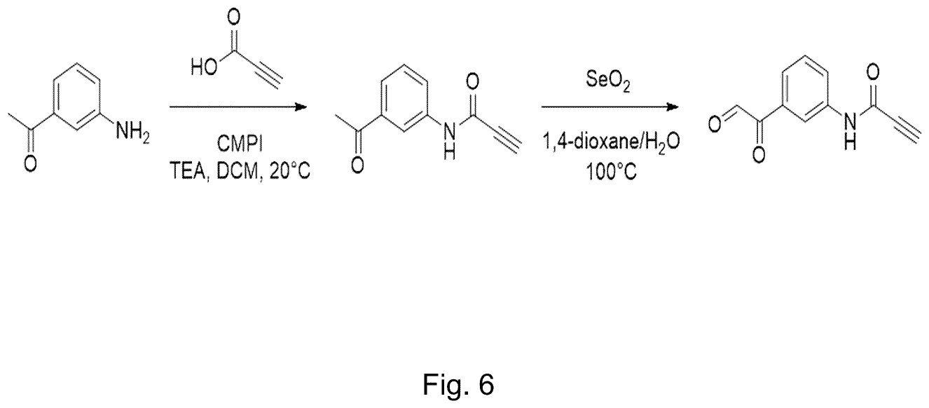

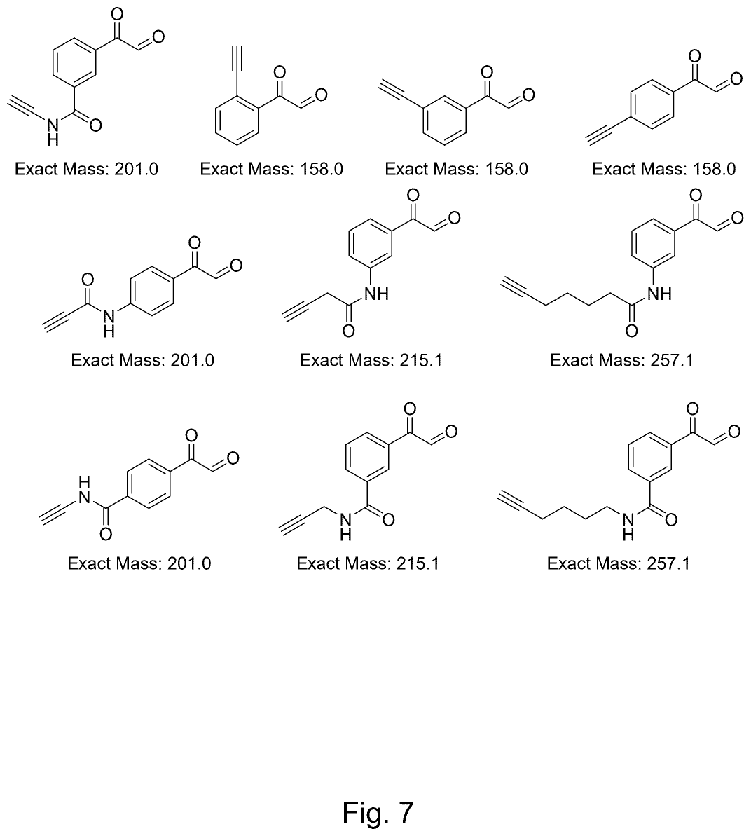

Exemplary Structure of PGA Tags—FIG. 6 shows a synthetic route of one specific PGA tag in an embodiment of the present invention, although multiple PGA tags with various structures are suitable for reacting with citrullinated peptides and other biomolecules containing ureido groups (see, as non-limiting examples, FIG. 7).

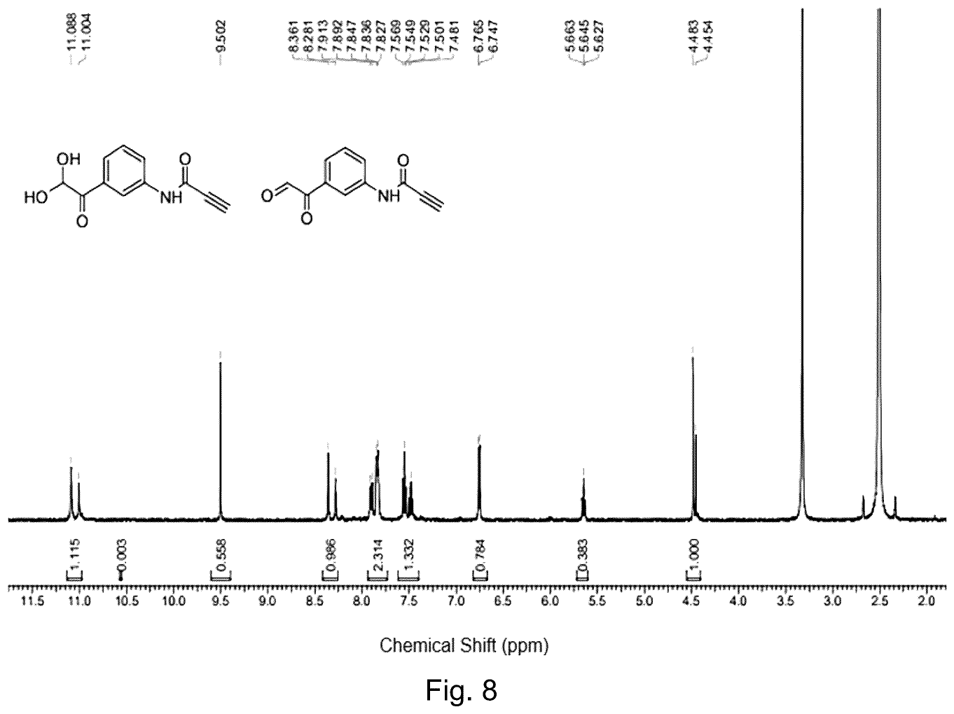

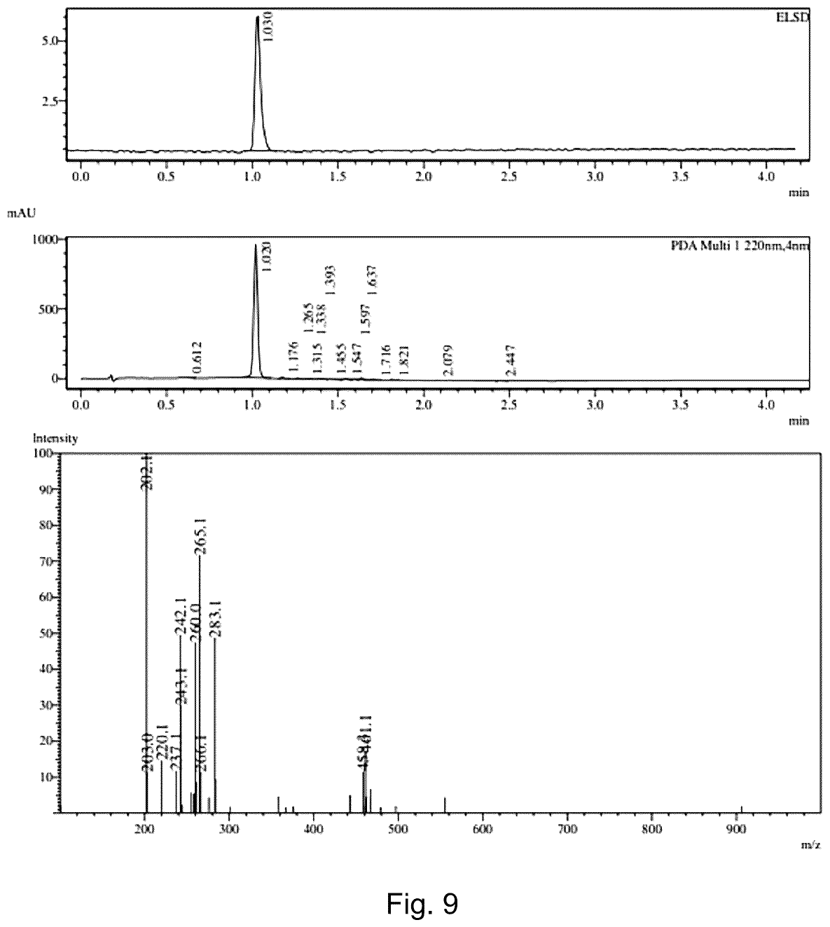

Characterization of PGA Tags—FIGS. 8 and 9 show the 1H-NMR and HPLC-UV-MS spectra of the PGA tag shown in FIG. 6. As seen in the NMR spectrum, the aldehyde and acetal forms of the compound convert to each other in DMSO.

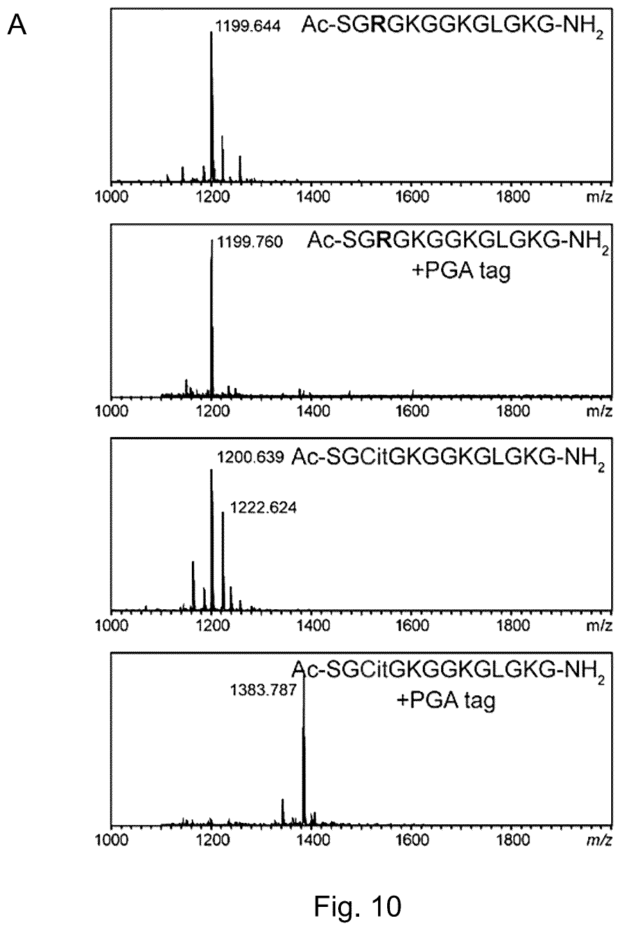

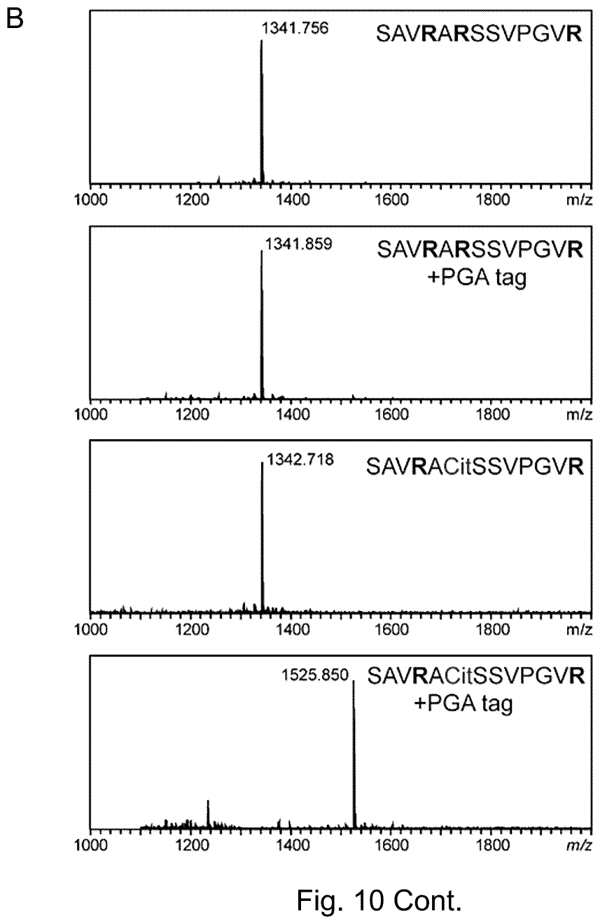

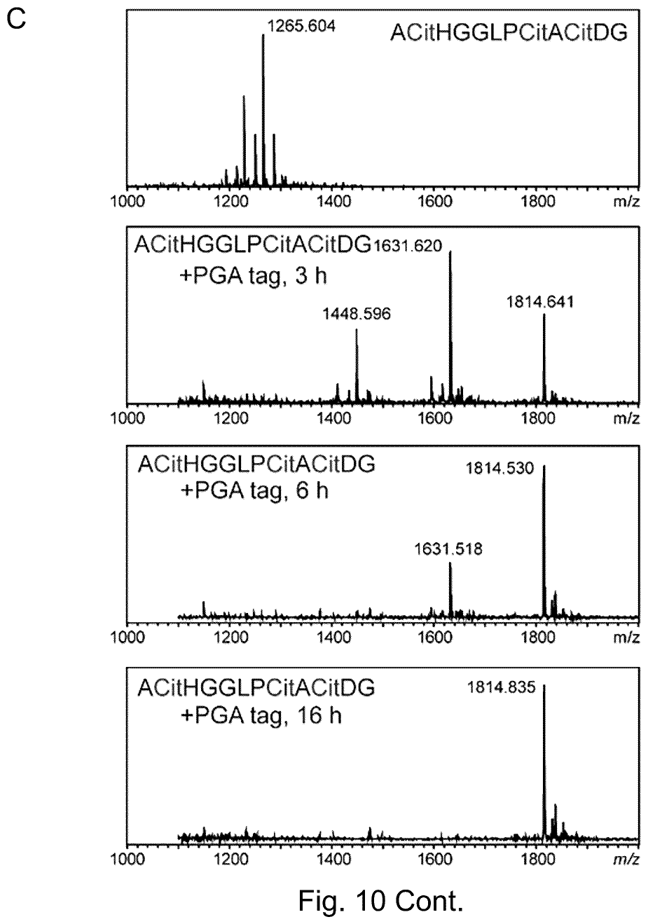

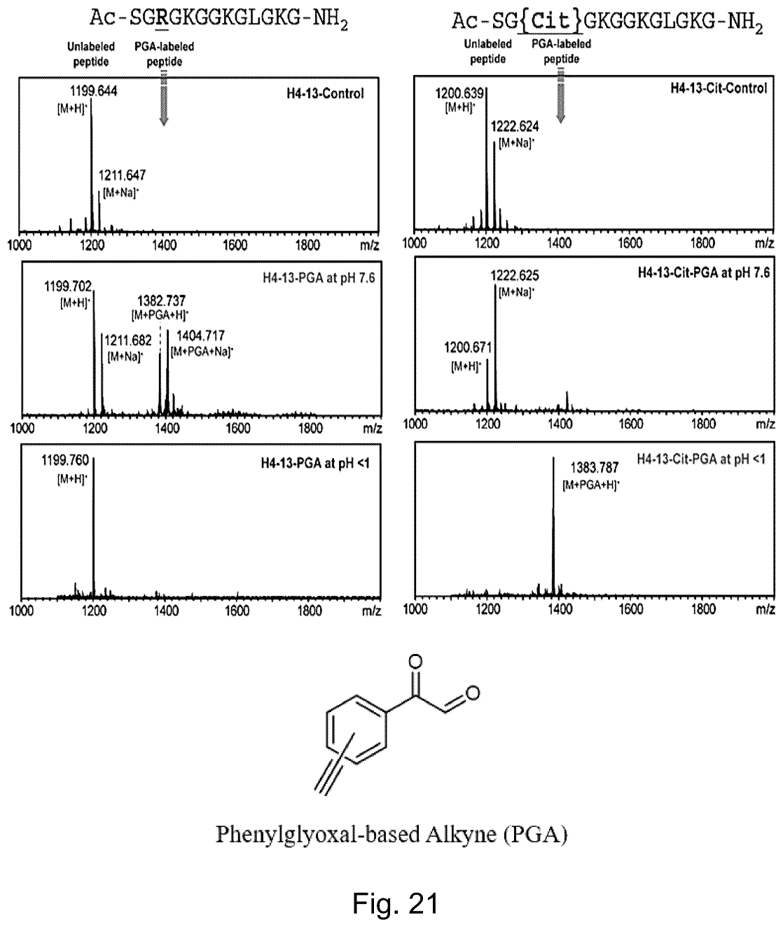

Chemical Selectivity of PGA to Citrullination—FIG. 10 shows MALDI-MS spectra of three different peptide standards (panel A-SGRGKGGKGLGKG (SEQ ID NO:1), panel B-SAVRARSSVPGVR (SEQ ID NO:2), and panel C-ARHGGLPRARDG (SEQ ID NO:3)) reacted with the PGA tag of FIG. 6 using acidic conditions under pH<1. The top row for FIG. 10 (panels A and B) shows the spectrum of the peptide standard (not citrullinated), the second row from the top shows the peptide standard (not citrullinated) mixed with the PGA tag, the third row shows the citrullinated peptide standard where the arginine residues (R) areconverted to citrulline (Cit), and the bottom row shows the citrullinated peptide standard reacted with the PGA tag. Each example was exposed to the acidic conditions. As seen in these figures, a significant change in mass of the primary peak did not occur until the citrullinated peptide standard was mixed with the PGA tag under acidic conditions, demonstrating the chemical specificity of the labeling reaction. FIG. 21 shows selective reactivity of a PGA tag with citrulline under acidic conditions (pH<1) compared to the peptide standard SGRGKGGKGLGKG (SEQ ID NO:1) and a mixture of the peptide and PGA tag at a pH of 7.6, as validated by histone standard peptide results.

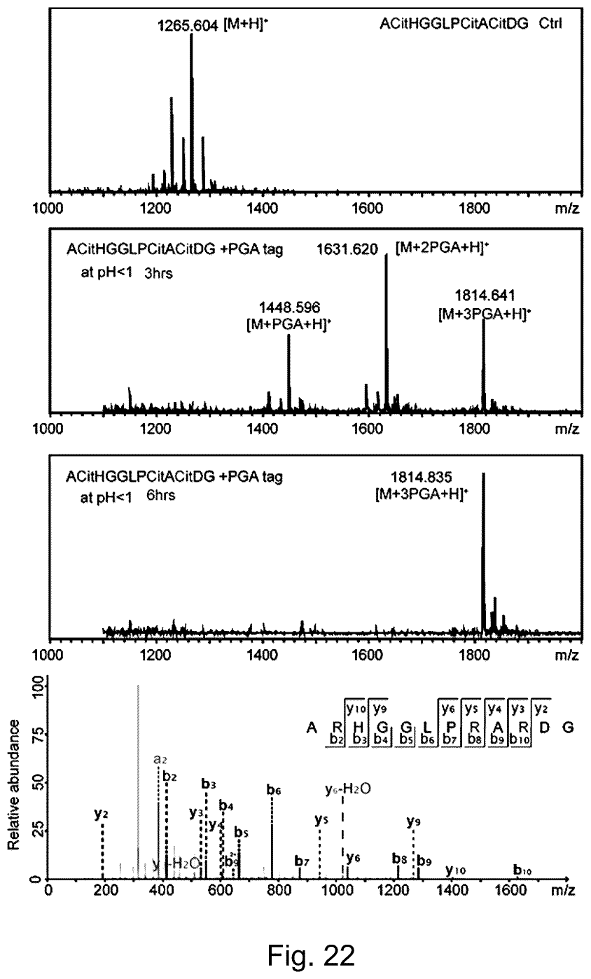

FIG. 10 (panel C) shows a citrullinated peptide standard (top row), and the citrullinated peptide standard mixed with the PGA tag after 3 hours (second row), 6 hours (third row), and 16 hours (bottom row). FIG. 22 shows probe concentrations and labeling times (3 hours and 6 hours). After a 6-hour incubation period, all three citrullination sites of the peptide ARHGGLPRARDG (SEQ ID NO:3) were fully labeled, resulting in more informative MS2 fragment ions (bottom panel).

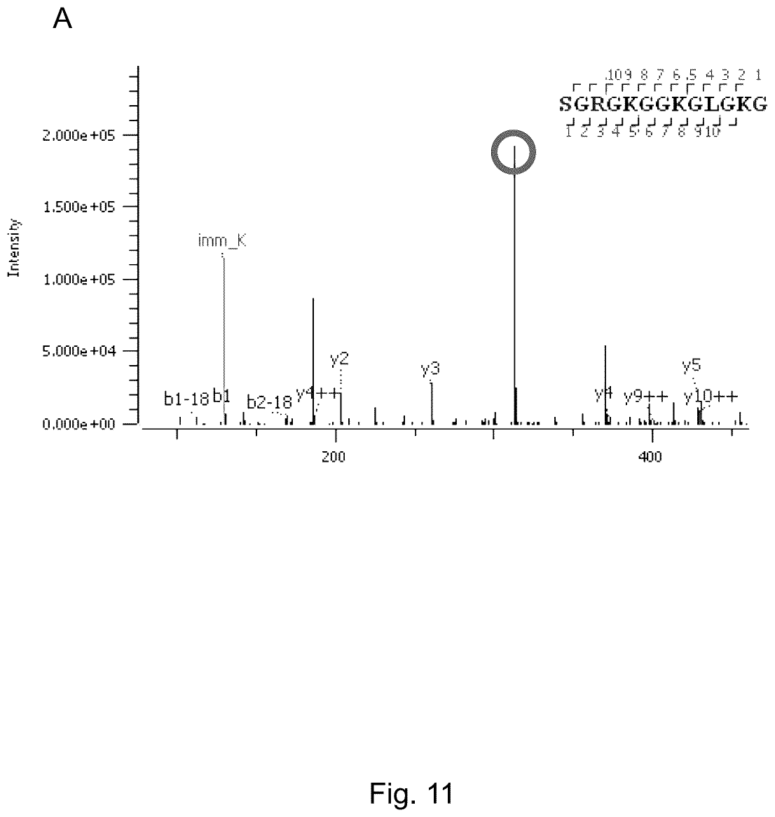

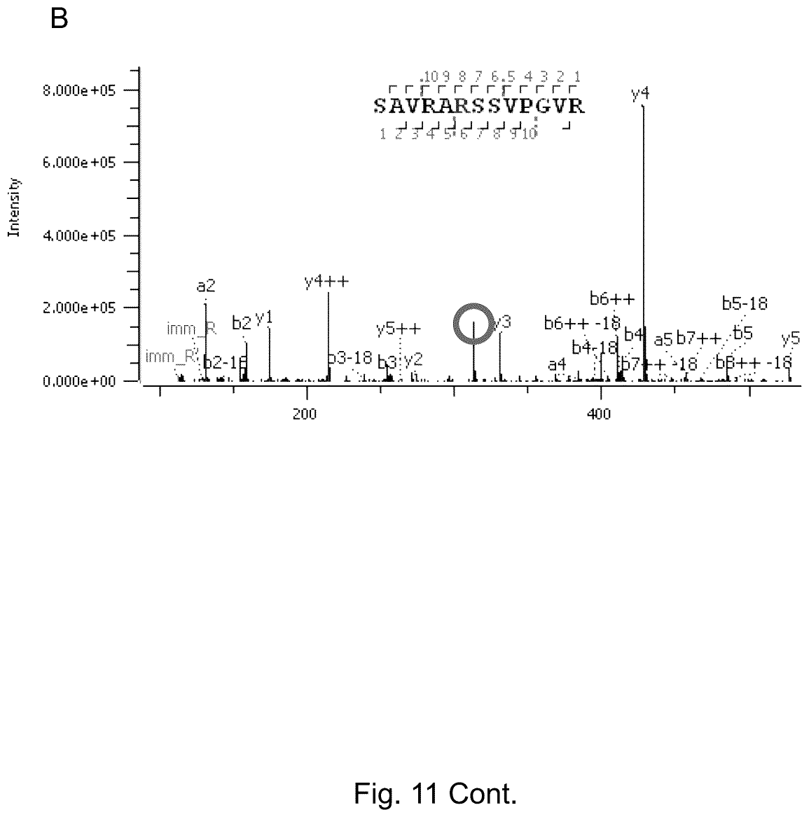

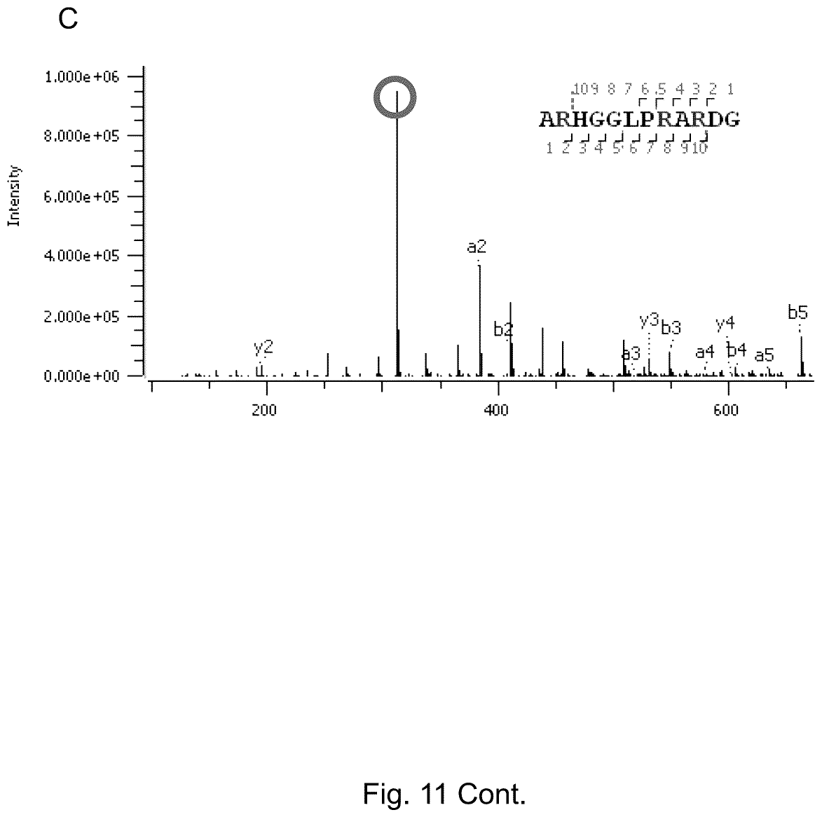

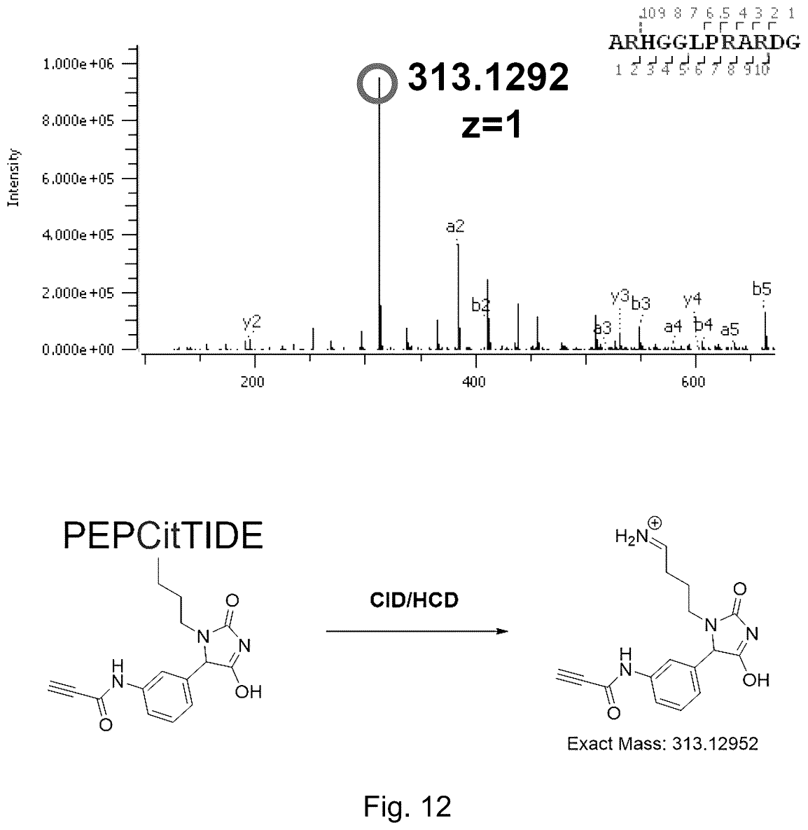

FIG. 11 (panels A-C) shows ESI-MS/MS spectra of different citrullinated peptides (panel A-SGRGKGGKGLGKG (SEQ ID NO:1), panel B-SAVRARSSVPGVR (SEQ ID NO:2), and panel C-ARHGGLPRARDG (SEQ ID NO:3)) labeled with the PGA tag of FIG. 8. FIG. 12 shows the MS/MS spectrum from FIG. 11 (panel C-ARHGGLPRARDG (SEQ ID NO:3)) where the signature immonium ion of the citrullinated peptide labeled with the PGA tag is seen at m/z 313. The easy identification of the signature fragment ion could facilitate fast scanning of citrullinated peptides.

Addition of Functional Tags Using Click Chemistry with PGA-Labeled Peptides—

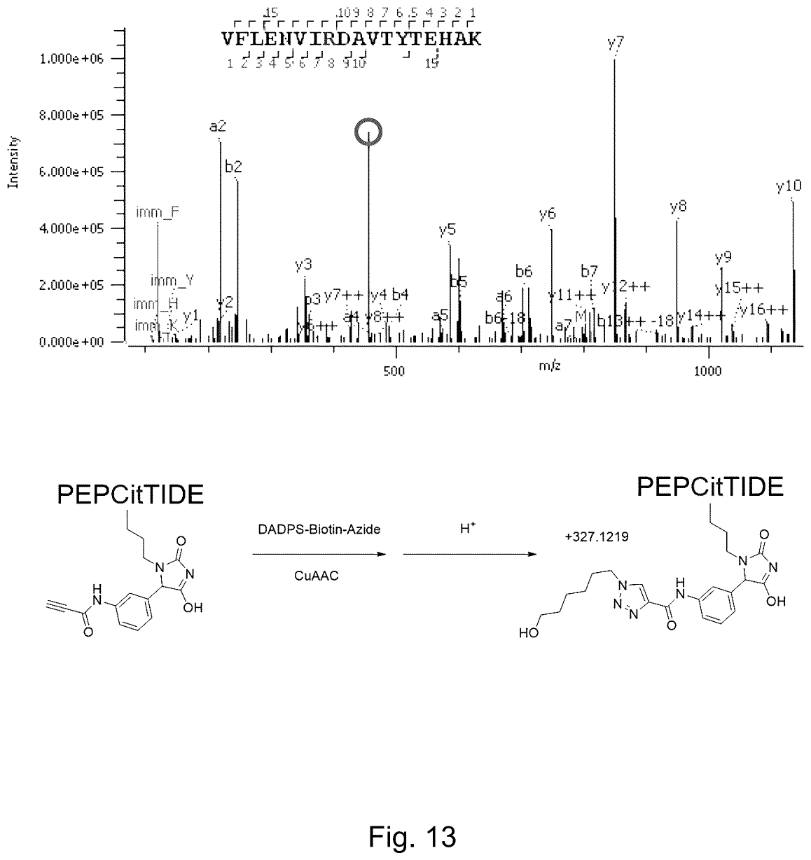

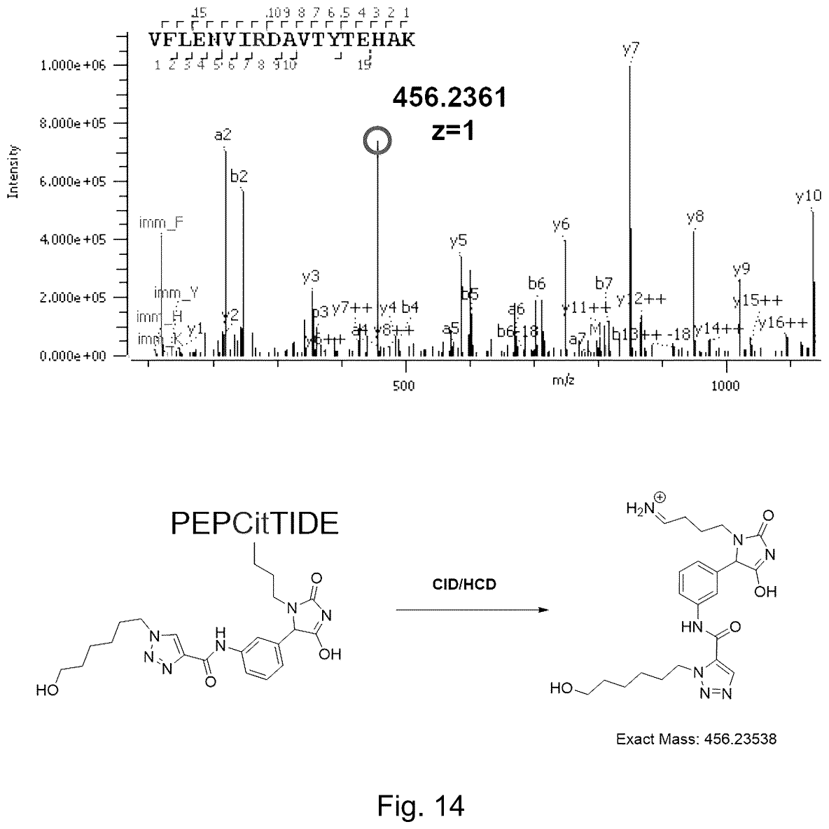

FIGS. 13 and 14 show ESI-MS/MS spectra of a citrullinated peptide (VFLENVIRDAVTYT EHAK (SEQ ID NO:4)) of Histone H4 labeled by the PGA tag and further labeled by a DADPS-Biotin-Azide functional tag. FIG. 14 shows the MS/MS spectrum from FIG. 13 where the signature immonium ion of the citrullinated peptide labeled with the PGA and azide tag is seen at m/z 456.

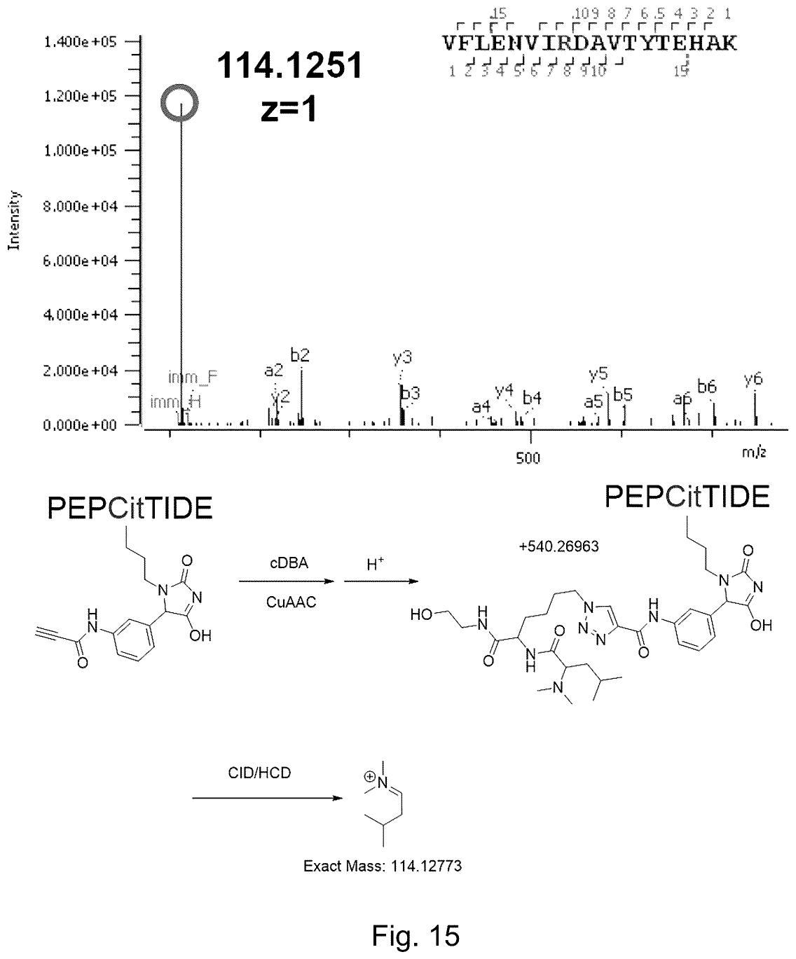

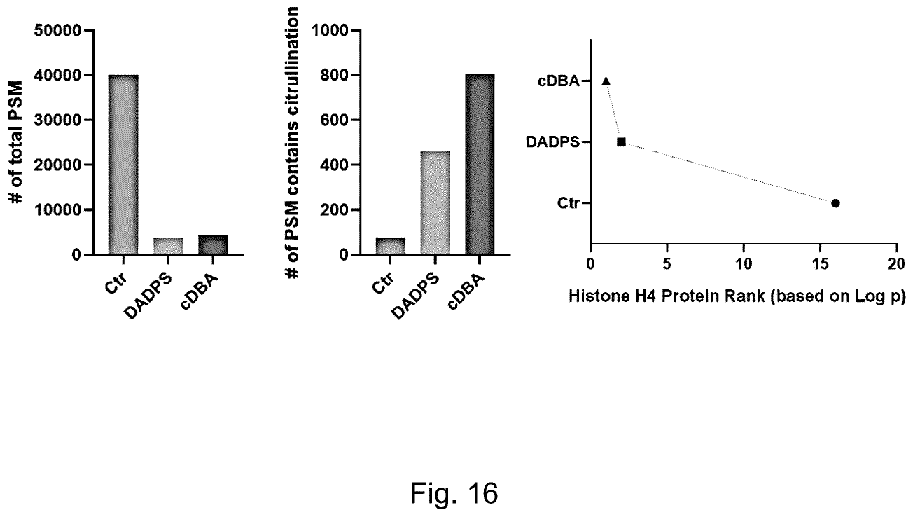

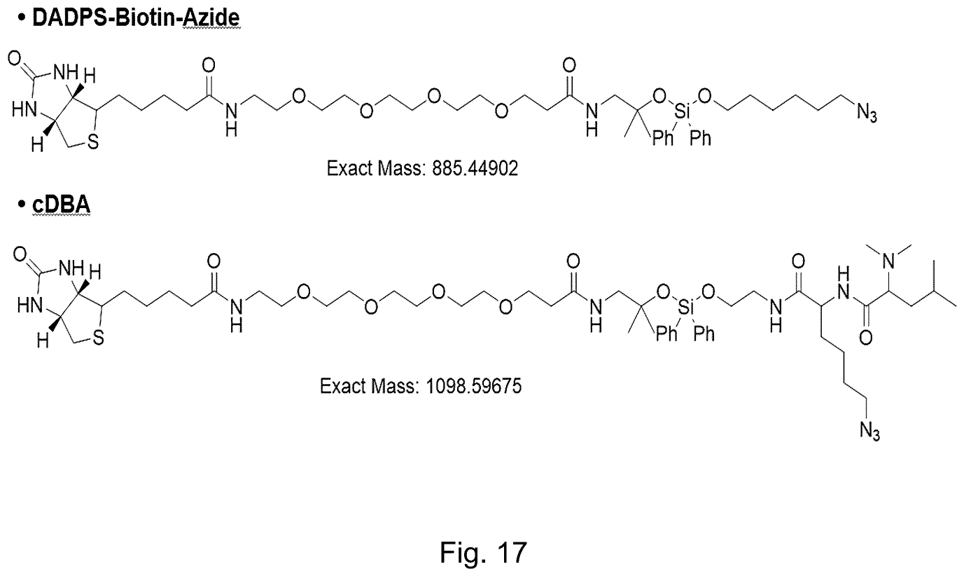

FIG. 15 shows ESI-MS/MS spectra of the VFLENVIRDAVTYTEHAK (SEQ ID NO: 4) citrullinated peptide labeled by PGA and further labeled by a cleavable DiLeu-Biotin-Azide (cDBA) tag. The signature immonium ion of the PGA-labeled peptide is not preserved but an abundant amount of the DiLeu reporter ion is seen. 10 μg of PGA-labeled Histone H4 peptides were mixed with 100 μg of Promega human digestion, followed by additional tagging with DADPS-Biton-Azide and cDBA tags, and followed by streptavidin enrichment (FIG. 16). Greater than 45× and 69× enrichment enhancement was achieved by the DADPS-Biton-Azide and cDBA tags, respectively. Structures of DADPS-biotin-azide and cDBA tags are illustrated in FIG. 17.

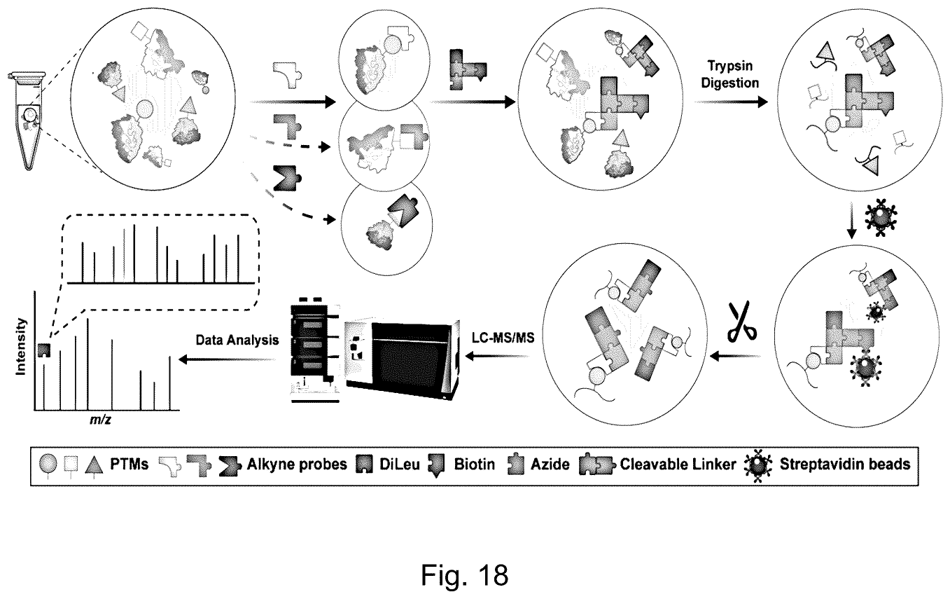

The PGA and DADPS-Biton-Azide and/or cDBA tags can further be combined as part of protein profiling platform called isobaric tandem orthogonal proteolysis activity-based protein profiling (isoBOP-ABPP), where proteins containing PTMs are labeled with PGA tags followed by DADPS-Biton-Azide and/or cDBA tags, digestion, enrichment and MS analysis (FIG. 18).

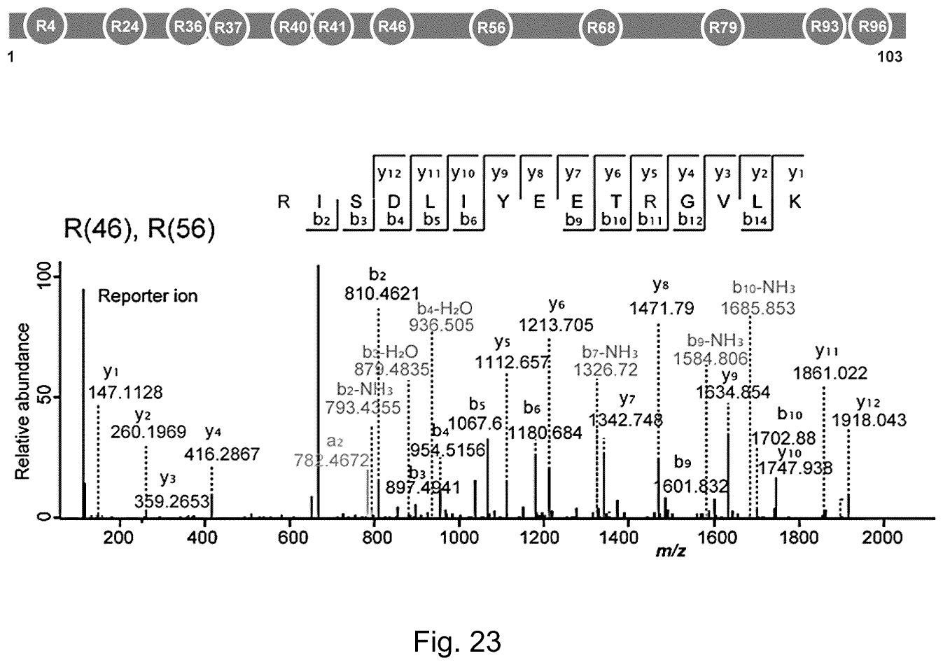

FIG. 23 shows 10 μg of citrullinated histone protein, including the fragment RISDLIYEETRGVLK (SEQ ID NO:5), being spiked into 1 mg of a complex proteome to evaluate the enrichment efficiency. With only one site not detected, all remaining 13 citrullination sites were successfully identified and quantified. Abundant b,y ions were produced to unambiguously pin-point Cit-sites. The PGA tag and isoBOP-ABPP platform provide the capability for multiple PTM analyses.

Comparisons With Existing Technologies—The PGA tags and methods of the present invention provide several advantages over conventional methods. Firstly, several commercially available anti-citrulline antibodies have been documented for use in immunochemistry (IHC) or enzyme-linked immunosorbent assay (ELISA) analyses targeting protein citrullination. However, these antibodies may either exhibit specificity towards specific proteins or serve for general citrullination level assessment. Moreover, conventional antibody-based detection methods fail to provide information regarding precise protein citrullination sites. Presently, no antibodies have been developed capable of effectively enriching all citrullinated peptides or proteins from intricate biological samples to facilitate large-scale citrullination analysis. The present PGA tags label citrullination sites based on the chemical selectivity between the phenylglyoxal group and the ureido group under acidic conditions. Therefore, it can profile global protein citrullination in an unbiased manner.

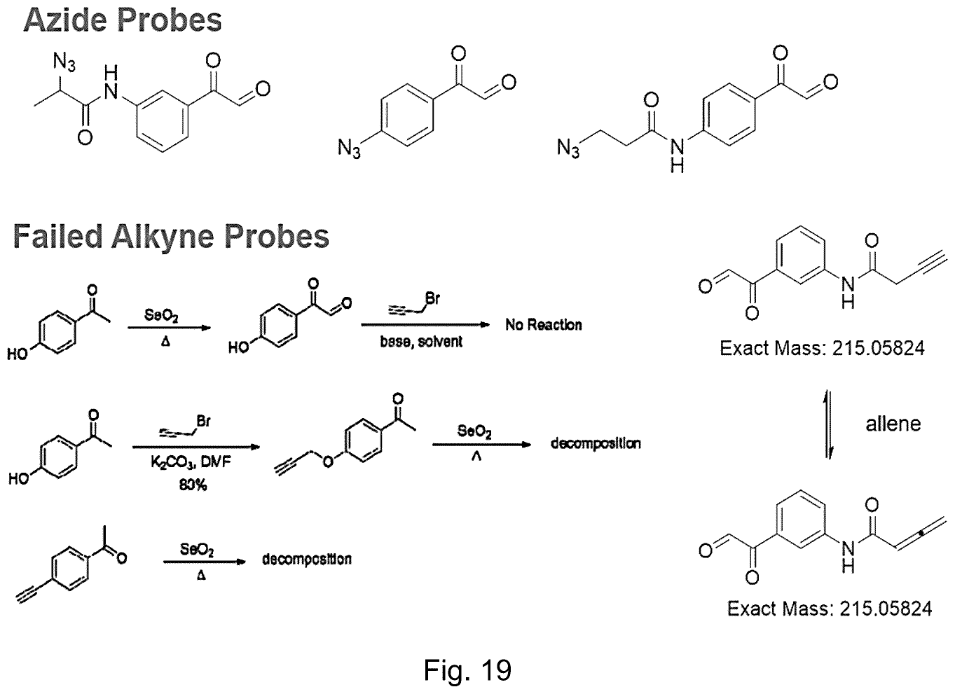

The PGA tags of the present invention are designed specifically for targeting citrullination sites. Despite the established recognition of the phenylglyoxal moiety for its reactivity with the ureido group, prior to this disclosure, there had been no successful creation of a phenylglyoxal-based alkyne tag capable of facilitating click chemistry-based chemical biology assays towards citrullination (see FIG. 19). This dearth of achievement may stem from inherent synthetic challenges and the potential instability associated with unoptimized structural configurations. For example, several attempts were previously made to synthesize alkyne-containing PG probes linked to reporter tags via the para position; however, none of these synthetic routes resulted in the desired tagging compounds (J Am Chem Soc. 2012 Oct. 17; 134 (41): 17015-17018). Furthermore, an additional carbon atom between the triple bond and the amide bond may cause the alkyne to switch to the corresponding allene compound. Phenylglyoxal-based azide probes have been previously disclosed (Molecules 2015, 20 (4), 6592-6600); however, the advantage of using an alkyne tag instead of an azide probe in chemical proteomics has been demonstrated to have lower background noise (Cell. 2020 Feb. 20; 180 (4): 605-632).

In contrast, the present PGA tags exhibit a compact structure and can be synthesized with high purity in a straightforward two-step process utilizing readily available, cost-effective commercial reagents. Their compact structure results in a relatively minor increase in mass (+226 Da, for example) upon conjugation to citrullination sites, thus minimizing potential interference due to bulky mass adduction. Furthermore, the simplicity of its synthesis renders the technology economically feasible for production and widely applicable in the field.

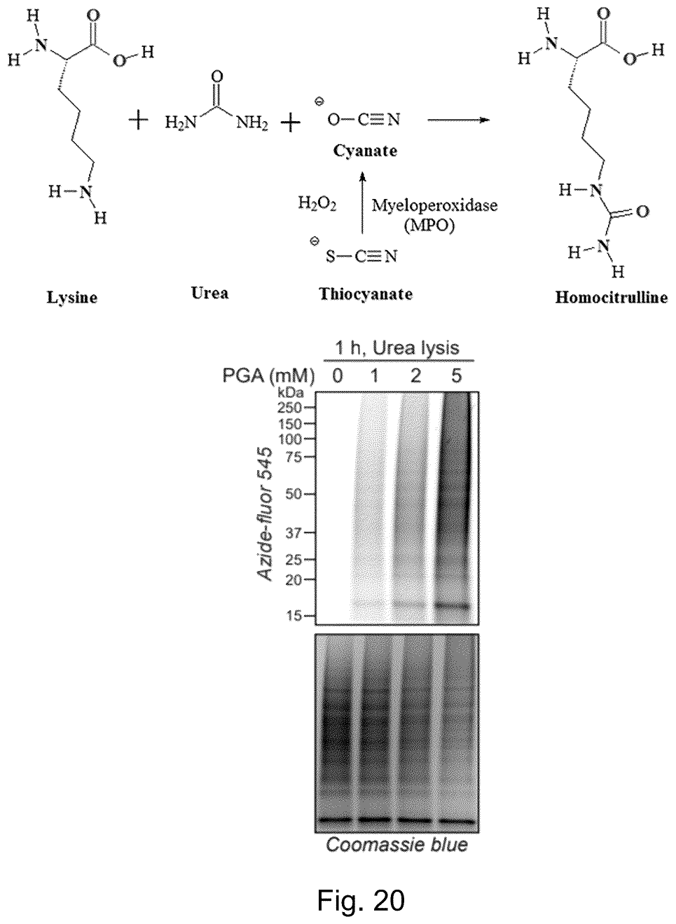

While the PGA tags have outstanding labeling efficiency towards citrullination sites, the labeling performance was investigated on citrullinated peptide standards as well as other proteins that carry the ureido group, including ureido groups present in carbamylation and homocitrullination (FIG. 20). Over 99% of the citrullination sites were completely labeled by PGA tag under optimized reaction conditions. Accordingly, the PGA tags of the present invention are also suitable for carbamlytion/homocitrullination analysis.

Citrullinated peptides labeled by PGA are able to be identified by tandem mass spectrometry. Abundant b/y ions are produced for site-specific identification of citrullination upon higher energy collisional dissociation (HCD) fragmentation. In addition, signature immonium ions are observed (at m/z 327, for example), which can be used as a diagnostic ion to quickly screen citrullinated peptides and improve identification accuracy.

Proteomic Analysis of Citrullination in Human Breast Cancer Cells—As an illustrative example, the application of PGA in conjunction with previously developed cleavable DiLeu-Biotin-Azide (cDBA) tags were demonstrated in high-throughput quantitative proteomic analysis of citrullination in human breast cancer cells.

PAD2 is one of the five PADs that catalyze the conversion of peptidyl-arginine to peptidyl-citrulline. Notably, PAD2 displays heightened expression levels in triple-negative breast cancer (TNBC) and HER2-positive breast cancer samples compared with normal samples. Additionally, PAD2 is involved in breast cancer growth and inhibition of PAD2 decreases disease progression and reduces tumor volume in vivo (Cancer Res. 2014; 74:6306-6317). However, the specific substrates targeted by PAD2 remain unexplored.

Using the PGA tag, and through integration with cleavable DiLeu-Biotin-Azide (cDBA) tag, high-throughput quantitative proteomic analysis was achieved of global citrullination in PAD2 knockdown TNBC cell lines. This innovation promises significant advancements in understanding PAD2-mediated citrullination and its role in breast cancer pathogenesis.

### Example 2—Characterization of PAD Enzymes Using PGA Probes and Construction of a Citrullination Site Library

To illustrate the extent of PGA probe integration with the isoBOP-ABPP approach, it was determined whether protein citrullination by PAD enzymes exhibited preferential substrate specificity. In vitro reactions were performed using four different PAD isozymes: PAD1, PAD2, PAD3, and PAD4. MDA-MB-231 cells (80% confluent) were washed with PBS and lysed on ice for 30 minutes in NP-40 lysis buffer containing protease and phosphatase inhibitors. Lysates were cleared by centrifugation (15,000×g, 15 min, 4° C.), and protein concentration was measured by BCA assay. One milligram of total protein was incubated with recombinant PAD enzymes (1:200, w/w) in citrullination buffer (HEPES, NaCl, CaCl₂), DTT) at 37° C. for 1 hour. Samples were reduced with TCEP (55° C., 1 h) and alkylated with iodoacetamide (room temp, 30 min, dark). Proteins were precipitated with methanol/chloroform, washed, and air-dried. Pellets were reconstituted in either SDS buffer for protein-centric labeling or RapiGest buffer for protease digestion and peptide-centric labeling.

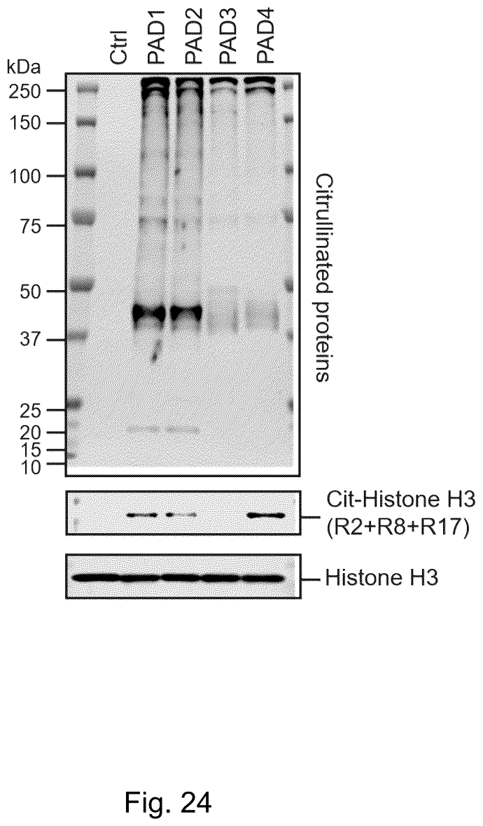

Significant differences were observed in the activity levels of the four PAD enzymes. Under the same reaction conditions, PAD1 and PAD2 exhibited relatively higher activity compared to PAD3 and PAD4 (see FIG. 24). Additionally, citrullination sites on Histone 3, specifically at R2, R8, and R17, were catalyzed exclusively by PAD1, PAD2 and PAD4. It is noteworthy that PAD4 catalyzed these sites on Histone 3 to a higher degree

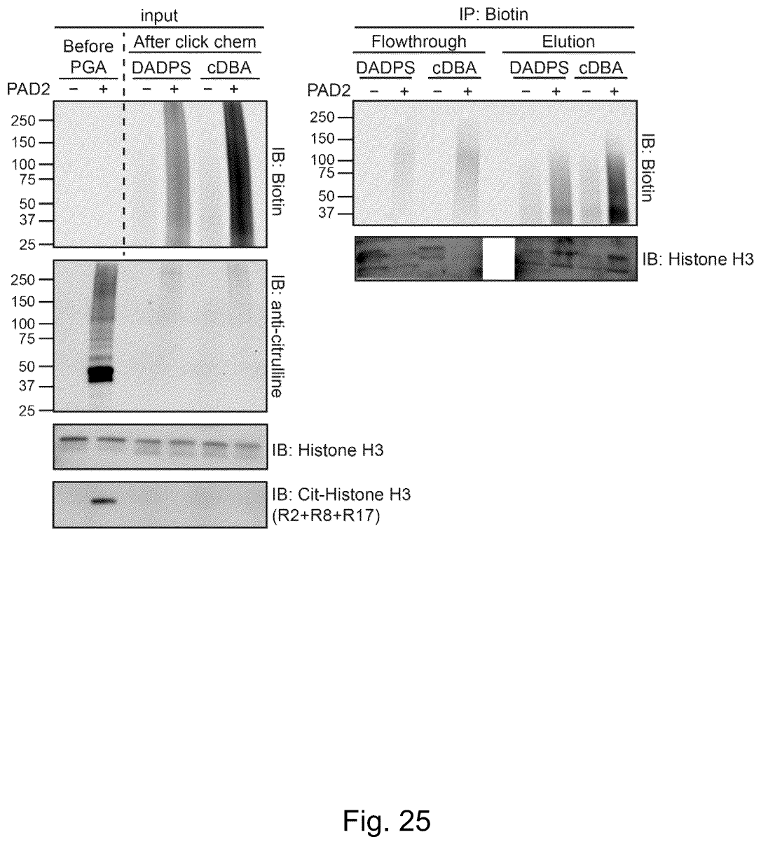

The specificity of the PGA probe was tested in complex samples, using PAD2 as an example. According to western blot results (FIG. 25), the PGA probe demonstrated specificity and a high level of enrichment when used in conjunction with the isoBOP-ABPP platform.

With the capability to achieve site-specific modifications established, this technology was used to examine the effects of auto-citrullination on PAD1-4 enzymes. Each PAD enzyme was incubated with an equal amount of BSA in reaction buffer (100 mM HEPES, pH 7.3, 50 mM NaCl, 10 mM CaCl₂), 2 mM DTT) at 37° C. for 1 hour, in triplicate for biological reproducibility. Reactions were analyzed using our peptide-centric workflow. Citrullination sites were classified as auto-citrullinated if their signal was significantly increased in the PAD-treated group compared to the control (fold change >2, p-value <0.05). Analysis of the protein 3D structures revealed that auto-citrullination sites clustered into three distinct regions: the C-terminal domain, subdomain 1, and subdomain 2. A substantial proportion of arginine residues in each PAD isoform were found to be auto-citrullinated. Specifically, 27 citrullinated residues were identified in PAD1 (76% of total arginines), 23 in PAD2 (61%), 19 in PAD3 (49%), and 15 in PAD4 (78%). These modifications showed strong overlap with previously reported auto-citrullination sites for each individual PAD enzyme.

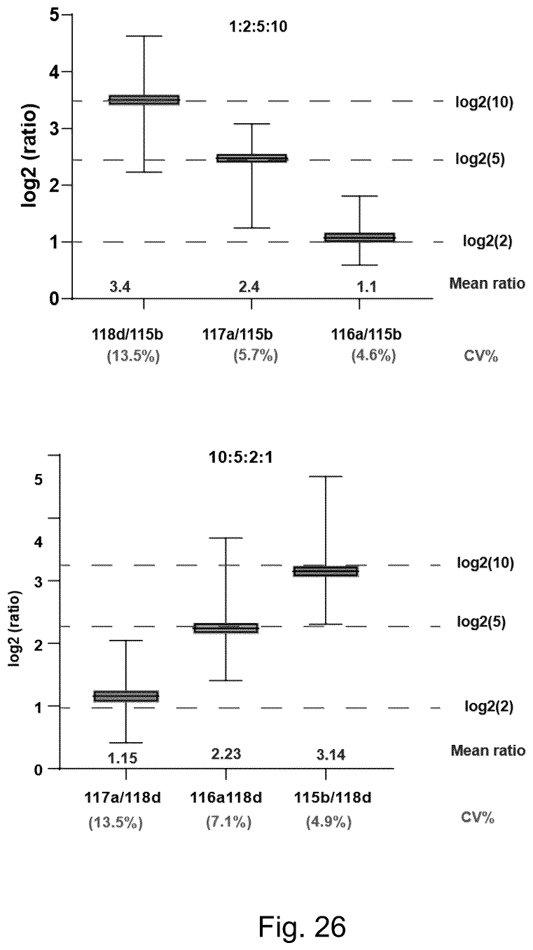

To assess quantification accuracy, four cDBA channels (115b, 116a, 117a, and 118d) were randomly selected and PGA-modified peptides were labelled according to a predefined ratio. As shown in FIG. 26, the measured intensities closely reflected the expected distribution, with an average coefficient of variation (CV) below 10%, indicating high precision and reproducibility of the quantification.

To construct a comprehensive human citrullination site library, in vitro PAD enzyme reactions were conducted using MDA-MB-231 cell pellets. Following trypsin digestion, peptides were labeled with the PGA probe as previously described. This approach enabled the quantification of 12,830 citrullinated sites across 3,389 proteins. Notably, while this total is slightly lower than that reported by Rebak et al. (Nature Structural & Molecular Biology, 2024, 31 (6): 977-995), this dataset was generated using only four LC fractions, in contrast to the 123 fractions employed in their study—highlighting the efficiency and depth of the present workflow. A single citrullination site was detected in 37.3% of the proteins, whereas 19.1% of the proteins contained more than five citrullinated arginine residues, indicating a broad spectrum of citrullination density across the proteome.

To identify substrate preferences of individual PAD enzymes, ANOVA analysis was performed on the quantified citrullination sites. A total of 12,221 sites showed statistically significant differences among PAD treatments (FDR<0.05). These sites were subsequently grouped into 14 clusters using K-nearest neighbors (KNN) clustering. To investigate sequence-specific substrate preferences of PAD enzymes, motif analysis was conducted on citrullinated sites from clusters that showed significant differences in the ANOVA-based clustering. The analysis revealed a strong enrichment of arginine residues flanked by aspartic acid (Asp), glycine (Gly), and serine (Ser), indicating these residues may play a role in PAD substrate recognition. This conserved motif highlights potential targets for the development of citrullination-specific antibodies with improved site selectivity.

The subcellular localization of the citrullinated substrates were analyzed to assess PAD enzyme preferences at the cellular level. The majority of substrates were localized to the cytoplasm and nucleus, consistent with previous reports. Additionally, it was observed that some citrullinated proteins were uniquely associated with a specific PAD enzyme treatment, while others were shared across two or more PAD-treated groups, suggesting both unique and overlapping substrate profiles among PAD family members. Gene Ontology (GO) biological process analysis was performed and significant enrichment was observed in processes related to translation, transcription, and RNA processing among the preferentially citrullinated substrates.

In summary, using the PGA tags and probes described herein, 12,830 citrullinated sites were quantified across 3,389 proteins, with 12,221 sites showing significant changes (FDR<0.05) for in vitro treatment of PAD isozymes. This large-scale data set highlights the specificity of PAD enzymes in recognizing distinct substrates.

A conserved citrullination motif was identified, enriched in arginine residues flanked by Glu, Lys, Arg and Ser, suggesting catalytic heterogeneity of PAD family. Understanding this motif not only provides insights into substrate recognition mechanisms but also enables the development of more selective tools, such as citrullination-specific antibodies, for studying PAD activity and its role in disease. This can be especially important for autoimmune diseases, such as rheumatoid arthritis, where PAD activity is implicated.

Most citrullinated proteins were localized to the cytoplasm and nucleus, consistent with existing literature. Some proteins were uniquely modified by specific PAD enzymes, while others were shared across multiple groups. The observation that some proteins were exclusively citrullinated by a specific PAD enzyme, while others were shared across multiple PAD groups, suggests that each PAD enzyme may be involved in distinct functional contexts within the cell. This variability also suggests that PAD enzymes can have overlapped and unique roles, depending on the cellular conditions and substrates present. The preferentially citrullinated proteins were significantly enriched in processes related to translation, transcription, and RNA processing.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

The invention claimed is:

1. A method of analyzing a target biomolecule in a sample, said method comprising the steps of:
a) providing a sample containing the target biomolecule, wherein the target biomolecule comprises a polypeptide having a region modified by a post-translational modification (PTM);
b) mixing a phenylglyoxal-based alkyne (PGA) tag with the target biomolecule to generate a labeled biomolecule, wherein the PGA tag is able to generate a derivatized polypeptide from the polypeptide having the region modified by the PTM.

2. The method of claim 1 further comprising: ionizing the labeled biomolecule to form a precursor ion; detecting and analyzing the precursor ion using a mass spectrometer; and identifying biomolecules with mass spectrometry data.

3. The method of claim 1, wherein the PGA tag comprises one or more of: 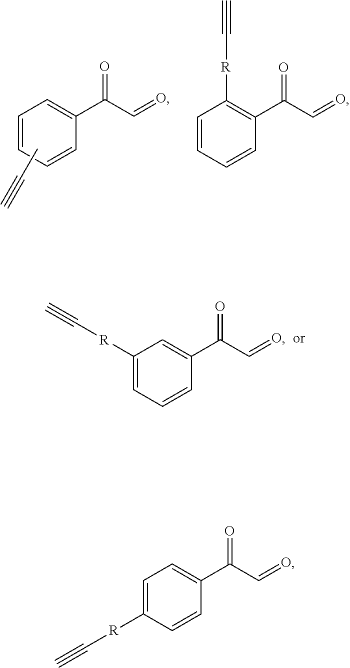
where R is selected from the group consisting of substituted and unsubstituted C₁ to C₂₀ alkylene groups and C₁ to C₂₀ amide groups.

4. The method of claim 3, wherein R is an amide having between 1-10 carbon atoms.

5. The method of claim 3, wherein the PGA tag comprises one or more of:

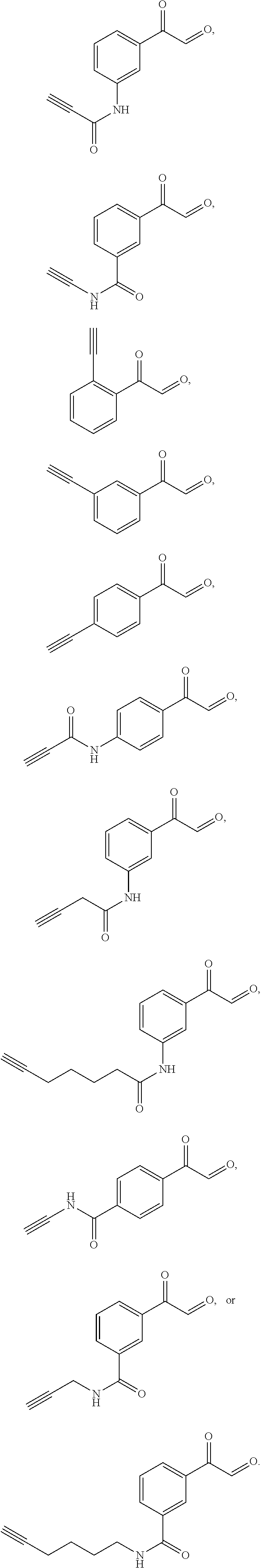

6. The method of claim 1, wherein the labeled biomolecule comprises an alkyne moiety and the method further comprises adding an additional functional tag to the labeled biomolecule, wherein the additional function tag comprises an azide group.

7. The method of claim 6, wherein the additional functional tag is a biotin tag comprising an azide group.

8. The method of claim 6, wherein the additional function tag is a DADPS-Biotin-Azide functional tag or a DiLeu-Biotin-Azide (cDBA) functional tag.

9. The method of claim 6, wherein the additional functional tag is an isotopically enriched functional tag comprising one or more heavy isotopes present in an amount in excess of the natural isotopic abundance.

10. The method of claim 6, further comprising:
providing two or more samples containing the target biomolecule;
in each of the two or more samples, adding the PGA tag with the target biomolecule to generate a labeled biomolecule, and adding the additional functional tag to the labeled biomolecule, wherein the additional functional tag in each sample is a different isotopically enriched functional tag, thereby generating two or more samples comprising different isotopically labeled target biomolecules;
combining the different isotopically labeled target biomolecules from the two or more samples and ionizing to form precursor ions; and
detecting and analyzing precursor ions from each of the two or more samples using a mass spectrometer.

11. The method of claim 6, wherein the step of adding an additional functional tag to the labeled biomolecule comprises performing a copper-catalyzed azide-alkyne cycloaddition (CuAAC) reaction.

12. The method of claim 1, wherein the PTM is selected from the group consisting of: citrullination, carbamylation, homocitrullination, and combinations thereof.

13. A method of analyzing a target biomolecule in a sample, said method comprising the steps of:
a) providing a sample containing the target biomolecule, wherein the target biomolecule comprises a ureido group;
b) mixing a phenylglyoxal-based alkyne (PGA) tag with the target biomolecule to generate a labeled biomolecule.

14. The method of claim 13 further comprising: ionizing the labeled biomolecule to form a precursor ion; detecting and analyzing the precursor ion using a mass spectrometer; and identifying biomolecules with mass spectrometry data.

15. The method of claim 13, wherein the PGA tag comprises one or more of: 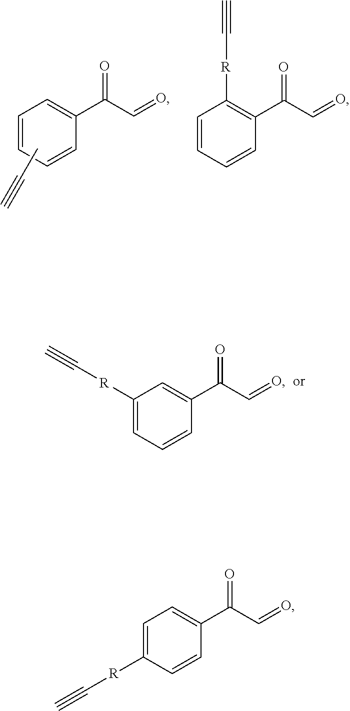
where R is selected from the group consisting of substituted and unsubstituted C₁ to C₂₀ alkylene groups and C₁ to C₂₀ amide groups.

16. The method of claim 13, wherein the PGA tag comprises one or more of:

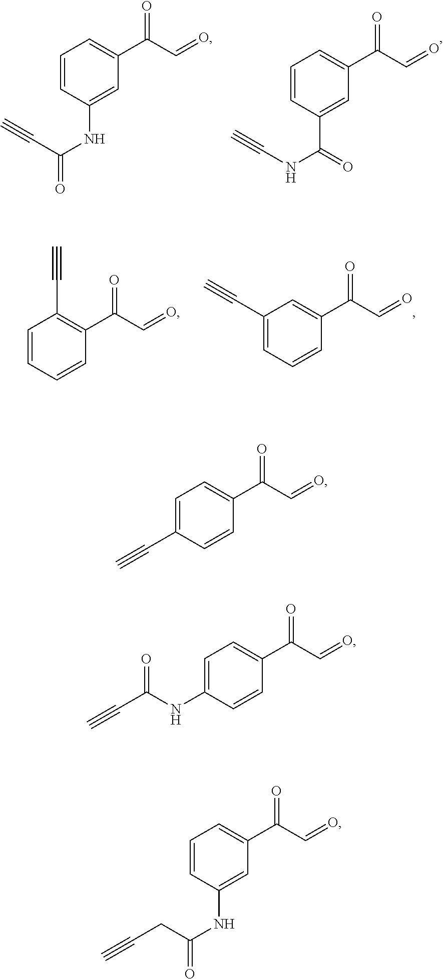

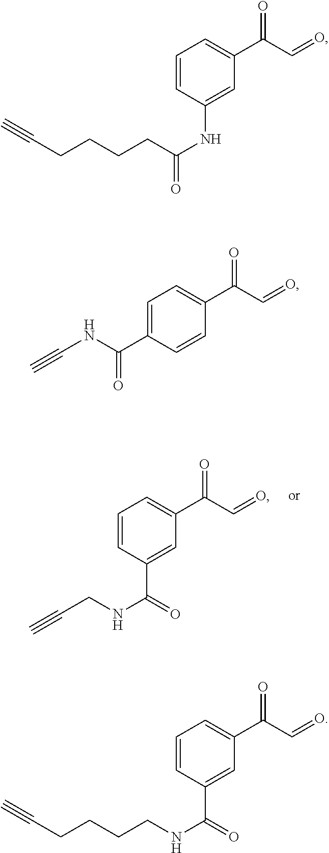 [SMILES: C#CCCCCC(=O)Nc1cccc(C(=O)C=O)c1 ; C#CNC(=O)c1ccc(C(=O)C=O)cc1 ; C#CCNC(=O)c1cccc(C(=O)C=O)c1 ; C#CCCCCNC(=O)c1cccc(C(=O)C=O)c1]

17. The method of claim 13, wherein the labeled biomolecule comprises an alkyne moiety and the method further comprises adding an additional functional tag to the labeled biomolecule, wherein the additional function tag comprises an azide group.

18. The method of claim 17, wherein the additional functional tag is a biotin tag comprising an azide group, or an isotopically enriched functional tag comprising one or more heavy isotopes present in an amount in excess of the natural isotopic abundance.

19. The method of claim 17, further comprising:
providing two or more samples containing the target biomolecule;
in each of the two or more samples, adding the PGA tag with the target biomolecule to generate a labeled biomolecule, and adding the additional functional tag to the labeled biomolecule, wherein the additional functional tag in each sample is a different isotopically enriched functional tag, thereby generating two or more samples comprising different isotopically labeled target biomolecules;
combining the different isotopically labeled target biomolecules from the two or more samples and ionizing to form precursor ions; and
detecting and analyzing precursor ions from each of the two or more samples using a mass spectrometer.

20. The method of claim 17, wherein the step of adding an additional functional tag to the labeled biomolecule comprises performing a copper-catalyzed azide-alkyne cycloaddition (CuAAC) reaction.


SEQUENCE LISTING (5 sequences)

SEQ ID NO: 1 - protein, 13 aa, synthetic construct
SGRGKGGKGLGKG

SEQ ID NO: 2 - protein, 13 aa, synthetic construct
SAVRARSSVPGVR

SEQ ID NO: 3 - protein, 12 aa, synthetic construct
ARHGGLPRARDG

SEQ ID NO: 4 - protein, 18 aa, synthetic construct
VFLENVIRDAVTYTEHAK

SEQ ID NO: 5 - protein, 15 aa, synthetic construct
RISDLIYEETRGVLK